(19) United States
(12) Patent Application Publication
(10) Pub. No.: US 2006/0167001 A1
(43) Pub. Date: Jul. 27, 2006

(54) Pyridazinone-derivatives as pde4 inhibitors

(76) Inventor: Jan Geert Sterk, UTRECHT (NL)

Correspondence Address: NATH & ASSOCIATES PLLC, 112 South West Street, Alexandria, VA, 22314, US

(21) Appl. No.: 10/523,112

(22) PCT Filed: Aug. 6, 2003
(86) PCT No.: PCT/EP03/08677

§ 371 Date: Feb. 3, 2005

(30) Foreign Application Priority Data
Aug. 10, 2002 (EP) 02017976.8

(51) Int. Cl. C07D 403/04; A61K 31/501

(52) U.S. Cl. 514/252.03; 544/238

(57) ABSTRACT

The compounds of a certain formula (1), 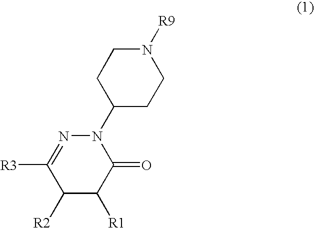 in which the given substituents have the meanings as given in the description, are novel effective PDE4 or PDE3/4 inhibitors.

### FIELD OF APPLICATION OF THE INVENTION

The invention relates to novel pyridazinone-derivatives, which are used in the pharmaceutical industry for the production of pharmaceutical compositions.

### KNOWN TECHNICAL BACKGROUND

International Patent Applications WO98/31674 (=U.S. Pat. No. 6,103,718), WO99/31071, WO99 31090, WO99/47505 (=U.S. Pat. No. 6,255,303), WO01/19818, WO01/30766, WO01/30777, WO01/94319, WO02/064584, WO02/085885 and WO02/085906 disclose phthalazinone derivatives having PDE4 inhibitory properties. In the International Patent Application WO94/12461 and in the European Patent Application EP 0 763 534 3-aryl-pyridazin-6-one and arylalkyl-diazinone derivatives are described as selective PDE4 inhibitors. International Patent Application WO93/07146 (=U.S. Pat. No. 5,716,954) discloses benzo and pyrido pyridazinone and pyridazinthione compounds with PDE4 inhibiting activity.

In the Journal of Medicinal Chemistry, Vol. 33, No. 6, 1990, pp. 1735-1741 1,4-Bis(3-oxo-2,3-dihydropyridazin-6-yl)benzene derivatives are described as potent phosphodiesterase inhibitors and inodilators. In the Journal of Medicinal Chemistry Vol. 45 No. 12, 2002, pp. 2520-2525, 2526-2533 and in Vol. 44, No. 16, 2001, pp. 2511-2522 and pp. 2523-2535 phthalazinone derivatives are described as selective PDE4 inhibitors.

### DESCRIPTION OF THE INVENTION

It has now been found that the pyridazinone-derivatives, which are described in greater details below, have surprising and particularly advantageous properties.

The invention thus relates to compounds of formula 1 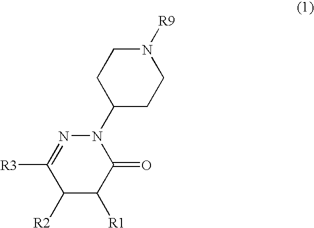
in which

R1 is hydrogen or 1-4C-alkyl,

R2 is hydrogen or 1-4C-alkyl,

R3 represents a phenyl derivative of formulae (a) or (b) 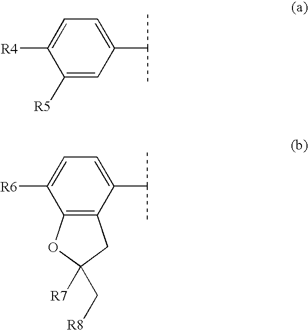
wherein
R4 is 1-4C-arloxy or 1-4C-alkoxy which is completely or predominantly substituted by fluorine,
R5 is 1-8C-alkoxy, 3-7C-cycloalkoxy, 3-7C-cycloalkylmethoxy, or 1-4C-alkoxy which is completely or predominantly substituted by fluorine,
R6 is 1-4C-alkoxy, 3-5C-cycloalkoxy, 3-5C-cycloalkylmethoxy, or 1-4C-alkoxy which is completely or predominantly substituted by fluorine,
R7 is 1-4C-alkyl and
R8 is hydrogen or 1-4C-alkyl,
or wherein
R7 and R8 together and with inclusion of the two carbon atoms, to which they are bonded, form a spiro-linked 5-, 6- or 7-membered hydrocarbon ring, optionally interrupted by an oxygen or sulphur atom,

R9 is 1-4C-alkyl, —S(O)₂—R10, —S(O)₂—(CH₂)ₙ—R11, —(CH₂)ₘ—S(O)₂—R12, —C((O)R13, —C(O)—(CH₂)ₙ—R14, —(CH₂)ₘ—C(O)—R15, Aryl1 or (Aryl2)-1-4C-alkyl,

R10 is 1-4C-alkyl, 5-dimethylaminonaphthalin-1-yl, —N(R16)R17, thiophenyl, phenyl or phenyl substituted by R18 and/or R19,

R11 is phenyl or —N(R16)R17,

R12 is —N(R16)R17,

R13 is 1-4C-alkyl, hydroxycarbonyl-1-4C-alkyl, phenyl, 2,4,6-trichlorophenyl, pyridyl, 4-ethyl-piperazin-2,3-dion-1-yl or -N(R16)R17,

R14 is —N(R16)R17,

R15 is —N(R16)R17, phenyl or phenyl substituted by R18 and/or R19 and/or R20,

R16 is hydrogen, 1-7C-alkyl, 3-7C-cycloalkyl, 3-7C-cycloalkylmethyl, phenyl or phenyl substituted by R18 and/or R19 and/or R20,

R17 1-7C-alkyl, 3-7C-cycloalkyl, 3-7C-cycloalkylmethyl, phenyl or phenyl substituted by R18 and/or R19 and/or R20,

or

R16 and R17 together and with inclusion of the nitrogen atom to which they are bonded, form a 4-morpholinyl-, 1-pyrrolidinyl-, 1-piperidinyl-, 1-hexahydroazepino- or a 1-piperazinyl-ring of formula (c) 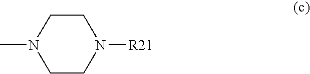
wherein
R21 is 1-4C-alkyl, pyrid-4-yl, pyrid-4-ylmethyl, 2-methoxyphenyl, 1,1-diphenylmethyl, dimethylamino-1-4C-alkyl, dimethylaminocarbonylmethyl, N-methyl-piperidin-4-yl, 4-morpholino-ethyl or tetrahydrofuran-2-ylmethyl,

R18 is halogen, nitro, cyano, carboxyl, 1-4C-alkyl, trifluoromethyl, 1-4C-alkoxy, 1-4C-alkoxy which is completely or predominantly substituted by fluorine, 1-4C-alkoxycarbonyl, amino, mono-or di-1-4C-alkylamino, aminocarbonyl, 1-4C-alkylcarbonylamino or mono-or di-1-4C-alkylaminocarbonyl,

R19 is halogen, amino, nitro, 1-4C-alkyl or 1-4C-alkoxy,

R20 is halogen,

Aryl1 is pyrimidin-2-yl, thieno-[2,3-d]pyrimidin-4-yl, 1-methyl-1H-pyrazolo-[3,4-d]pyrimidin-4-yl, thiazoyl, imidazolyl, furanyl, pyridyl, phenyl or phenyl substituted by R18 and/or R19,

Aryl2 is pyridyl, phenyl, phenyl substituted by R18 and/or R19, 2-oxo-2H-chromen-7-yl or 4-(1,2,3-thiadiazol-4-yl)phenyl,

n is an integer from 1 to 4,

m is an integer from 1 to 4,

and the salts of these compounds.

1-4C-Alkyl is a straight-chain or branched alkyl radical having 1 to 4 carbon atoms. Examples are the butyl, isobutyl, sec-butyl, tert-butyl, propyl, isopropyl, ethyl and methyl radicals.

1-7C-Alkyl is a straight-chain or branched alkyl radical having 1 to 7 carbon atoms. Examples are the heptyl, isoheptyl(5-methylhexyl), hexyl, isohexyl(4-methylpentyl), neohexyl(3,3-dimethylbutyl), pentyl, isopentyl(3-methylbutyl), neopentyl(2,2-dimethylpropyl), butyl, isobutyl, sec-butyl, tert-butyl, propyl, isopropyl, ethyl and methyl radicals.

1-4C-Alkoxy is a radical which, in addition to the oxygen atom, contains a straight-chain or branched alkyl radical having 1 to 4 carbon atoms. Alkoxy radicals having 1 to 4 carbon atoms which may be mentioned in this context are, for example, the butoxy, isobutoxy, sec-butoxy, tert-butoxy, propoxy, isopropoxy, ethoxy and methoxy radicals.

1-8C-Alkoxy is a radical which, in addition to the oxygen atom, contains a straight-chain or branched alkyl radical having 1 to 8 carbon atoms. Alkoxy radicals having I to 8 carbon atoms which may be mentioned in this context are, for example, the octyloxy, heptyloxy, isoheptyloxy(5-methylhexyloxy), hexyloxy, isohexyloxy(4-methylpentyloxy), neohexyloxy(3,3-dimethylbutoxy), pentyloxy, isopentyloxy(3-methylbutoxy), neopentyloxy(2,2-dimethylpropoxy), butoxy, isobutoxy, sec-butoxy, tert-butoxy, propoxy, isopropoxy, ethoxy and methoxy radicals.

Halogen within the meaning of the present invention is bromine, chlorine or fluorine.

3-7C-Cycloalkoxy stands for cyclopropyloxy, cyclobutyloxy, cyclopentyloxy, cyclohexyloxy or cycloheptyloxy, of which cyclopropyloxy, cyclobutyloxy and cyclopentyloxy are preferred.

3-7C-Cycloalkylmethoxy stands for cyclopropylmethoxy, cyclobutylmethoxy, cyclopentylmethoxy, cyclohexylmethoxy or cycloheptylmethoxy, of which cyclopropylmethoxy, cyclobutylmethoxy and cyclopentylmethoxy are preferred.

3-5C-Cycloalkoxy stands for cyclopropyloxy, cyclobutyloxy and cyclopentyloxy.

3-5C-Cycloalkylmethoxy stands for cyclopropylmethoxy, cyclobutylmethoxy and cyclopentylmethoxy.

3-7C-Cycloalkyl stands for cyclopropyl, cyclobutyl, cyclopentyl, cyclohexyl or cycloheptyl, of which cyclopropyl and cyclopentyl are preferred

3-7C-Cycloalkylmethyl stands for cyclopropylmethyl, cyclobutylmethyl, cyclopentylmethyl, cyclohexylmethyl or cycloheptylmethyl.

1-4C-Alkoxy which is completely or predominantly substituted by fluorine is, for example, the 2,2,3,3,3-pentafluoropropoxy, the perfluoroethoxy, the 1,2,2-trifluoroethoxy and in particular the 1,1,2,2-tetrafluoroethoxy, the 2,2,2-trifluoroethoxy, the trifluoromethoxy and the difluoromethoxy radical, of which the difluoromethoxy radical is preferred. “Predominantly” in this connection means that more than half of the hydrogen atoms of the 1-4C-alkoxy group are replaced by fluorine atoms.

As spiro-linked 5-, 6- or 7-membered hydrocarbon rings, optionally interrupted by an oxygen or sulphur atom, may be mentioned the cyclopentane, cyclohexane, cycloheptane, tetrahydrofuran, tetrahydropyran and the tetrahydrothiophen ring.

1-4C-Alkoxycarbonyl is a carbonyl group to which one of the abovementioned 1-4C-alkoxy radicals is bonded. Examples are the methoxycarbonyl [CH₃O—C(O)—] and the ethoxycarbonyl [CH₃CH₂O—C(O)—] radical.

An 1-4C-Alkylcarbonylamino radical is, for example, the propionylamino [C₃H₇C(O)NH—] and the acetylamino radical [CH₃C(O)NH—].

Mono- or Di-1-4C-alkylamino radicals contain in addition to the nitrogen atom, one or two of the above-mentioned 1-4C-alkyl radicals. Preferred are the di-1-4C-alkylamino radicals, especially the dimethylamino, the diethylamino and the diisopropylamino radical.

Mono- or Di-1-4C-alkylaminocarbonyl radicals contain in addition to the carbonyl group one of the abovementioned mono- or di-1-4C-alkylamino radicals. Examples which may be mentioned are the N-methyl- the N,N-dimethyl-, the N-ethyl-, the N-propyl-, the N,N-diethyl- and the N-isopropylaminocarbonyl radical.

(Aryl2)-1-4C-alkyl radicals stand for one of the abovementioned 1-4C-alkyl radicals substituted by an Aryl2 radical. Examples which may be mentioned are the pyrid-3-ylmethyl, pyrid-4-ylmethyl or benzyl radical.

Hydroxycarbonyl-1-4C-alkyl stand for one of the abovementioned 1-4C-alkyl radicals substituted by a hydroxycarbonyl (carboxyl) radical.

Dimethylamino-1-4C-alkyl radicals stand for one of the abovementioned 1-4C-alkyl radicals substituted by a dimethylamino radical.

Suitable salts for compounds of the formula 1 are all acid addition salts. Particular mention may be made of the pharmacologically tolerable inorganic and organic acids customarily used in pharmacy. Those suitable are water-soluble and water-insoluble acid addition salts with acids such as, for example, hydrochloric acid, hydrobromic acid, phosphoric acid, nitric acid, sulphuric acid, acetic acid, citric acid, D-gluconic acid, benzoic acid, 2-(4-hydroxybenzoyl)benzoic acid, butyric acid, sulphosalicylic acid, maleic acid, lauric acid, malic acid, fumaric acid, succinic acid, oxalic acid, tartaric acid, embonic acid, stearic acid, toluenesulphonic acid, methanesulphonic acid or 3-hydroxy-2-naphthoic acid, the acids being employed in salt preparation—depending on whether a mono- or polybasic acid is concerned and depending on which salt is desired—in an equimolar quantitative ratio or one differing therefrom.

Pharmacologically intolerable salts, which can be obtained, for example, as process products during the preparation of the compounds according to the invention on an industrial scale, are converted into pharmacologically tolerable salts by processes known to the person skilled in the art.

According to expert's knowledge the compounds of the invention as well as their salts may contain, e.g. when isolated in crystalline form, varying amounts of solvents. Included within the scope of the invention are therefore all solvates and in particular all hydrates of the compounds of formula 1 as well as all solvates and in particular all hydrates of the salts of the compounds of formula 1.

Compounds of formula 1 to be emphasized are those in which

R1 is hydrogen,

R2 is hydrogen or 1-4C-alkyl,

R3 represents a phenyl derivative of formulae (a) or (b) 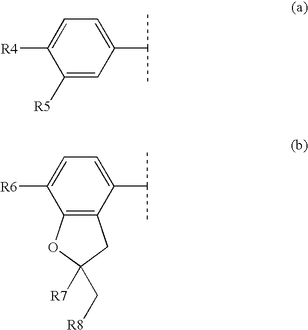
wherein
R4 is 1-2C-alkoxy or 1-2C-alkoxy which is completely or predominantly substituted by fluorine,
R5 is 1-4C-alkoxy,
R6 is 1-2C-alkoxy or 1-2C-alkoxy which is completely or predominantly substituted by fluorine,
R7 is methyl and
R8 is hydrogen,
or wherein
R7 and R8 together and with inclusion of the two carbon atoms, to which they are bonded, form a spiro-linked cyclopentane, cyclohexane, tetrahydrofurane or tetrahydropyran ring,

R9 is 1-4C-alkyl, —S(O)₂—R10, —S(O)₂—(CH₂)ₙ—R11, —C(O)R13, —C(O)—(CH₂)ₙ—R14, —(CH₂)ₘ—C(O)—R15 or (Aryl2)-1-4C-alkyl,
R10 is 1-4C-alkyl, 5-dimethylaminonaphthalin-1-yl, —N(R16)R17, thiophenyl, phenyl or phenyl substituted by R18 and/or R19,

R11 is phenyl,

R13 is 1-4C-alkyl, phenyl, pyridyl, 2,4,6-trichlorophenyl, 4-ethyl-piperazin-2,3-dion-1-yl or —N(R16)R17,

R14 is —N(R16)R17,

R15 is —N(R16)R17, phenyl or phenyl substituted by R18 and/or R19,

R16 is hydrogen,1-7C-alkyl, 3-7C-cycloalkyl or 3-7C-cycloalkylmethyl,

R17 is 1-7C-alkyl, 3-7C-cycloalkyl or 3-7C-cycloalkylmethyl,

or

R16 and R17 together and with inclusion of the nitrogen atom to which they are bonded, form a 4-morpholinyl-, 1-pyrrolidinyl-, 1-piperidinyl-, 1-hexahydroazepino- or a 1-piperazinyl-ring of formula (c) 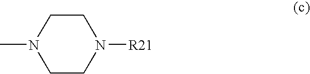
wherein
R21 is 1-4C-alkyl, pyrid-4-yl, 2-methoxyphenyl, 1,1-diphenylmethyl or N-methyl-piperidin-4-yl,

R18 is halogen, nitro, cyano, 1-4C-alkyl, trifluoromethyl, 1-4C-alkoxy, 1-4C-alkoxy which is completely or predominantly substituted by fluorine or 1-4C-alkoxycarbonyl,

R19 is halogen, amino, nitro, 1-4C-alkyl or 1-4C-alkoxy,

Aryl2 is pyridyl, phenyl or phenyl substituted by R18 and/or R19,

m is an integer from 1 to 2,

n is an integer from 1 to 2,

and the salts of these compounds.

Preferred compounds of formula 1 are those, in which

R1 is hydrogen,

R2 is hydrogen or methyl,

R3 represents a phenyl derivative of formula (a) 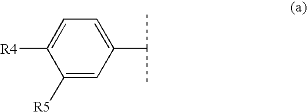
wherein
R4 is 1-2C-alkoxy,
R5 is 1-2C-alkoxy,

R9 is —S(O)₂—R10, —S(O)₂—(CH₂)ₙ—R11, —C(O)R13, —C(O)—(CH₂)ₙ—R14, —(CH₂)ₘ—C(O)—R15 or (Aryl2)-1-2C-alkyl,

R10 is 1-4C-alkyl, 5-dimethylaminonaphthalin-1-yl, —N(R16)R17, thiophenyl, phenyl or phenyl substituted by R18 and/or R19,

R11 is phenyl,

R13 is 1-4C-alkyl, phenyl, 2,4,6-trichlorophenyl, pyridyl, 4-ethyl-piperazin-2,3-dion-1-yl or —N(R16)R17,

R14 is —N(R16)R17,

R15 is —N(R16)R17,

R16 is hydrogen or 1-4C-alkyl,

R17 is 1-4C-alkyl,

or

R16 and R17 together and with inclusion of the nitrogen atom to which they are bonded, form a 4-morpholinyl-, 1-pyrrolidinyl-, 1-piperidinyl- or a 1-piperazinyl-ring of formula (c) 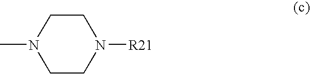
wherein
R21 is 1-4C-alkyl, pyrid-4-yl, 2-methoxyphenyl or 1,1-diphenylmethyl,

R18 is halogen, cyano, 1-4C-alkyl, trifluoromethyl, 1-4C-alkoxy or 1-4C-alkoxy which is completely or predominantly substituted by fluorine,

R19 is 1-4C-alkyl or 1-4C-alkoxy,

Aryl2 is pyridyl or phenyl,

m is 1,

n is 1,

and the salts of these compounds.

Particularly preferred compounds of formula 1 are those in which

R1 is hydrogen,

R2 is hydrogen or methyl,

R3 represents a phenyl derivative of formula (a) 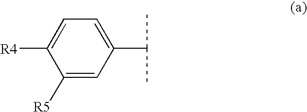
wherein
R4 is methoxy,
R5 is methoxy and
R9 is acetyl, morpholin-4-ylcarbonyl, pyridin-3-ylmethyl, 4-ethyl-piperazin-2,3-dion-1-ylcarbonyl, 4-methylpiperazin-1-ylcarbonyl, 5-dimethylamino-naphythalene-1-sulfonyl, 2-(morpholin-4-yl)-2-oxo-ethyl, 4-methylbenzenesulfonyl, methylsulfonyl, 4-chlorobenzenesulfonyl, benzylsulfonyl, 4-methoxybenzenesulfonyl, benzenesulfonyl, 2,5-dimethoxybenzenesulfonyl, 2-cyanobenzenesulfonyl, thiophen-2-ylsulfonyl, 2-fluorobenzenesulfonyl, 2-trifluoromethoxy-benzenesulfonyl, dimethylaminosulfonyl, benzoyl, pyridin-3-ylcarbonyl, 2,4,6-trichlorobenzenecarbonyl, tert-butylaminocarbonyl, dimethylaminocarbonylmethyl, 2-(4-methyl-piperazin-1-yl)-2-oxo-ethyl, 2-(4-pyridin-4-ylpiperazin-1-yl)ethanoyl, 2-[4-(2-methoxyphenyl)piperazin-1-yl]ethanoyl or 2-[4-(1,1-diphenylmethyl)piperazin-1-yl]ethanoyl,

and the salts of these compounds.

An embodiment (embodiment A) of the compounds of formula 1 are those in which

R1 is hydrogen or 1-4C-alkyl,

R2 is hydrogen or 1-4C-alkyl,

R3 represents a phenyl derivative of formulae (a) or (b) 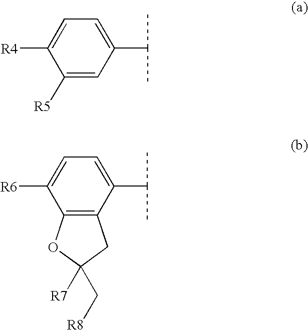
wherein
R4 is 1-4C-alkoxy or 1-4C-alkoxy which is completely or predominantly substituted by fluorine,
R5 is 1-8C-alkoxy, 3-7C-cycloalkoxy, 3-7C-cycloalkylmethoxy, or 1-4C-alkoxy which is completely or predominantly substituted by fluorine,
R6 is 1-4C-alkoxy, 3-5C-cycloalkoxy, 3-5C-cycloalkylmethoxy, or 1-4C-alkoxy which is completely or predominantly substituted by fluorine,
R7 is 1-4C-alkyl and
R8 is hydrogen or 1-4C-alkyl,
or wherein
R7 and R8 together and with inclusion of the two carbon atoms, to which they are bonded, form a spiro-linked 5-, 6- or 7-membered hydrocarbon ring, optionally interrupted by an oxygen or sulphur atom,

R9 is —S(O)₂—R10, —S(O)₂—(CH₂)ₙ—R11 or —(CH₂)ₘ—S(O)₂—R12,

R10 is 1-4C-alkyl, 5-dimethylaminonaphthalin-1-yl, -N(R16)R17, thiophenyl, phenyl or phenyl substituted by R18 and/or R19,

R11 is phenyl or —N(R16)R17,

R12 is —N(R16)R17,

R16 is hydrogen, 1-7C-alkyl, 3-7C-cycloalkyl, 3-7C-cycloalkylmethyl, phenyl or phenyl substituted by R18 and/or R19 and/or R20,

R17 1-7C-alkyl, 3-7C-cycloalkyl, 3-7C-cycloalkylmethyl, phenyl or phenyl substituted by R18 and/or R19 and/or R20,

or

R16 and R17 together and with inclusion of the nitrogen atom to which they are bonded, form a 4-morpholinyl-, 1-pyrrolidinyl-, 1-piperidinyl-, 1-hexahydroazepino- or a 1-piperazinyl-ring of formula (c) 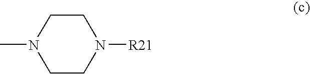
wherein
R21 is 1-4C-alkyl, pyrid-4-yl, pyrid-4-ylmethyl, 2-methoxyphenyl, 1,1-diphenylmethyl, dimethylamino-1-4C-alkyl, dimethylaminocarbonylmethyl, N-methyl-piperidin-4-yl, 4-morpholino-ethyl or tetrahydrofuran-2-ylmethyl,

R18 is halogen, nitro, cyano, carboxyl, 1-4C-alkyl, trifluoromethyl, 1-4C-alkoxy, 1-4C-alkoxy which is completely or predominantly substituted by fluorine, 1-4C-alkoxycarbonyl, amino, mono-or di-1-4C-alkylamino, aminocarbonyl, 1-4C-alkylcarbonylamino or mono-or di-1-4C-alkylaminocarbonyl,

R19 is halogen, amino, nitro, 1-4C-alkyl or 1-4C-alkoxy,

R20 is halogen,

n is an integer from 1 to 4,

m is an integer from 1 to 4,

and the salts of these compounds.

Preferred compounds of formula 1 of embodiment A are those in which

R1 is hydrogen,

R2 is hydrogen or methyl,

R3 represents a phenyl derivative of formula (a) 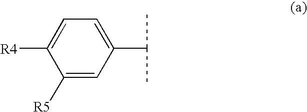
wherein
R4 is 1-2C-alkoxy,
R5 is 1-2C-alkoxy,
R9 is —S(O)₂—R10 or —S(O)₂—(CH₂)ₙ—R11,

R10 is 1-4C-alkyl, 5-dimethylaminonaphthalin-1-yl, —N(R16)R17, thiophenyl, phenyl or phenyl substituted by R18 and/or R19,

R11 is phenyl,

R16 is hydrogen or 1-4C-alkyl,

R17 is 1-4C-alkyl,

R18 is halogen, cyano, 1-4C-alkyl, trifluoromethyl, 1-4C-alkoxy or 1-4C-alkoxy which is completely or predominantly substituted by fluorine,

R19 is 1-4C-alkyl or 1-4C-alkoxy,

n is 1,

and the salts of these compounds.

Particularly preferred compounds of formula 1 of embodiment A are those in which

R1 is hydrogen,

R2 is hydrogen or methyl,

R3 represents a phenyl derivative of formula (a) 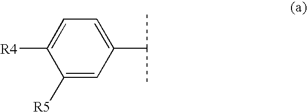
wherein
R4 is methoxy,
R5 is methoxy and

R9 is 5-dimethylamino-naphythalene-1-sulfonyl, 4-methylbenzenesulfonyl, methylsulfonyl, 4-chlorobenzenesulfonyl, benzylsulfonyl, 4-methoxybenzenesulfonyl, benzenesulfonyl, 2,5-dimethoxybenzenesulfonyl, 2-cyanobenzenesulfonyl, thiophen-2-ylsulfonyl, 2-fluorobenzenesulfonyl, 2-trifluoromethoxy-benzenesulfonyl or dimethylaminosulfonyl,

and the salts of these compounds.

Another embodiment (embodiment B) of the compounds of formula 1 are those in which

R1 is hydrogen or 1-4C-alkyl,

R2 is hydrogen or 1-4C-alkyl,

R3 represents a phenyl derivative of formulae (a) or (b) 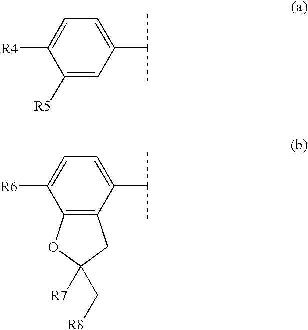
wherein
R4 is 1-4C-alkoxy or 1-4C-alkoxy which is completely or predominantly substituted by fluorine,
R5 is 1-8C-alkoxy, 3-7C-cycloalkoxy, 3-7C-cycloalkylmethoxy, or 1-4C-alkoxy which is completely or predominantly substituted by fluorine,
R6 is 1-4C-alkoxy, 3-5C-cycloalkoxy, 3-5C-cycloalkylmethoxy, or 1-4C-alkoxy which is completely or predominantly substituted by fluorine,
R7 is 1-4C-alkyl and
R8 is hydrogen or 1-4C-alkyl,
or wherein
R7 and R8 together and with inclusion of the two carbon atoms, to which they are bonded, form a spiro-linked 5-, 6- or 7-membered hydrocarbon ring, optionally interrupted by an oxygen or sulphur atom,

R9 is —C(O)R13, —C(O)—(CH₂)ₙ—R14 or —(CH₂)ₘ—C(O)—R15,

R13 is 1-4C-alkyl, hydroxycarbonyl-1-4C-alkyl, phenyl, 2,4,6-trichlorophenyl, pyridyl, 4-ethyl-piperazin-2,3-dion-1-yl or —N(R16)R17,

R14 is —N(R16)R17,

R15 is —N(R16)R17, phenyl or phenyl substituted by R18 and/or R19 and/or R20,

R16 is hydrogen, 1-7C-alkyl, 3-7C-cycloalkyl, 3-7C-cycloalkylmethyl, phenyl or phenyl substituted by R18 and/or R19 and/or R20,

R17 1-7C-alkyl, 3-7C-cycloalkyl, 3-7C-cycloalkylmethyl, phenyl or phenyl substituted by R18 and/or R19 and/or R20,

or

R16 and R17 together and with inclusion of the nitrogen atom to which they are bonded, form a 4-morpholinyl-, 1-pyrrolidinyl-, 1-piperidinyl-, 1-hexahydroazepino- or a 1-piperazinyl-ring of formula (c) 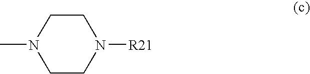
wherein
R21 is 1-4C-alkyl, pyrid-4-yl, pyrid-4-ylmethyl, 2-methoxyphenyl, 1,1-diphenylmethyl, dimethylamino-1-4C-alkyl, dimethylaminocarbonylmethyl, N-methyl-piperidin-4-yl, 4-morpholino-ethyl or tetrahydrofuran-2-ylmethyl,

R18 is halogen, nitro, cyano, carboxyl, 1-4C-alkyl, trifluoromethyl, 1-4C-alkoxy, 1-4C-alkoxy which is completely or predominantly substituted by fluorine, 1-4C-alkoxycarbonyl, amino, mono-or di-1-4C-alkylamino, aminocarbonyl, 1-4C-alkylcarbonylamino or mono-or di-1-4C-alkylaminocarbonyl,

R19 is halogen, amino, nitro, 1-4C-alkyl or 1-4C-alkoxy,

R20 is halogen,

n is an integer from 1 to 4,

m is an integer from 1 to 4,

and the salts of these compounds.

Preferred compounds of formula 1 of embodiment B are those in which

R1 is hydrogen,

R2 is hydrogen or methyl,

R3 represents a phenyl derivative of formula (a) 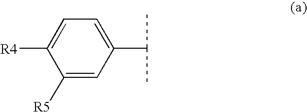
wherein
R4 is 1-2C-alkoxy,
R5 is 1-2C-alkoxy,

R9 is —C(O)R13, —C(O)—(CH₂)ₙ—R14 or —(CH₂)ₘ—C(O)—R15,

R13 is 1-4C-alkyl, phenyl, 2,4,6-trichlorophenyl, pyridyl, 4-ethyl-piperazin-2,3-dion-1-yl or —N(R16)R17,

R14 is —N(R16)R17,

R15 is —N(R16)R17,

R16 is hydrogen or 1-4C-alkyl,

R17 is 1-4C-alkyl,

or

R16 and R17 together and with inclusion of the nitrogen atom to which they are bonded, form a 4-morpholinyl-, 1-pyrrolidinyl-, 1-piperidinyl- or a 1-piperazinyl-ring of formula (c) 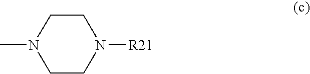
wherein
R21 is 1-4C-alkyl, pyrid-4-yl, 2-methoxyphenyl or 1,1-diphenylmethyl,

m is1,

n is 1,

and the salts of these compounds.

Particularly preferred compounds of formula 1 of embodiment B are those in which

R1 is hydrogen,

R2 is hydrogen or methyl,

R3 represents a phenyl derivative of formula (a) 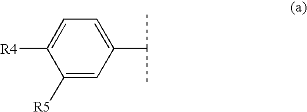
wherein
R4 is methoxy,
R5 is methoxy and

R9 is acetyl, morpholin-4-ylcarbonyl, 4-ethyl-piperazin-2,3-dion-1-ylcarbonyl, 4-methylpiperazin-1-ylcarbonyl, 2-(morpholin-4-yl)-2-oxo-ethyl, benzoyl, pyridin-3-ylcarbonyl, 2,4,6-trichlorobenzenecarbonyl, tert-butylaminocarbonyl, dimethylaminocarbonylmethyl, 2-(4-methyl-piperazin-1-yl)-2-oxo-ethyl, 2-(4-pyridin-4-ylpiperazin-1-yl)ethanoyl, 2-[4-(2-methoxyphenyl)piperazin-1-yl]ethanoyl or 2-[4-(1,1-diphenylmethyl)piperazin-1-yl]ethanoyl,

and the salts of these compounds.

Further compounds of formula 1 (embodiment C) are those in which

R1 is hydrogen or 1-4C-alkyl,

R2 is hydrogen or 1-4C-alkyl,

R3 represents a phenyl derivative of formulae (a) or (b) 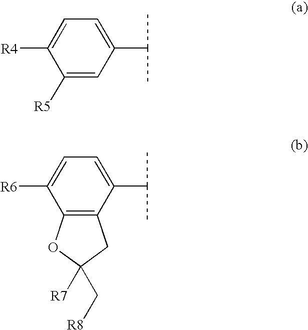
wherein
R4 is 1-4C-alkoxy or 1-4C-alkoxy which is completely or predominantly substituted by fluorine,
R5 is 1-8C-alkoxy, 3-7C-cycloalkoxy, 3-7C-cycloalkylmethoxy, or 1-4C-alkoxy which is completely or predominantly substituted by fluorine,
R6 is 1-4C-alkoxy, 3-5C-cycloalkoxy, 3-5C-cycloalkylmethoxy, or 1-4C-alkoxy which is completely or predominantly substituted by fluorine,
R7 is 1-4C-alkyl and
R8 is hydrogen or 1-4C-alkyl,
or wherein
R7 and R8 together and with inclusion of the two carbon atoms, to which they are bonded, form a spiro-linked 5-, 6- or 7-membered hydrocarbon ring, optionally interrupted by an oxygen or sulphur atom,

R9 is hydrogen, 1-4C-alkyl, —S(O)₂—R10, —S(O)₂—(CH₂)ₙ—R11, —(CH₂)ₘ—S(O)₂—R12, —C(O)R13, —C(O)—(CH₂)ₙ—R14, —(CH₂)ₘ—C(O)—R51, Aryl1 or (Aryl2)-1-4C-alkyl,

R10 is 1-4C-alkyl, 5-dimethylaminonaphthalin-1-yl, -N(R16)R17, phenyl or phenyl substituted by R18 and/or R19,

R11 is —N(R16)R17,

R12 is —N(R16)R17,

R13 is 1-4C-alkyl, hydroxycarbonyl-1-4C-alkyl, phenyl, pyridyl, 4-ethyl-piperazin-2,3-dion-1-yl or —N(R16)R17,

R14 is —N(R16)R17,

R15 is —N(R16)R17, phenyl or phenyl substituted by R18 and/or R19 and/or R20,

R16 and R17 are independent from each other hydrogen, 1-7C-alkyl, 3-7C-cycloalkyl, 3-7C-cycloalkyl-methyl, phenyl or phenyl substituted by R18 and/or R19 and/or R20, or R16 and R17 together and with inclusion of the nitrogen atom to which they are bonded, form a 4-morpholinyl-, 1-pyrrolidinyl-, 1-piperidinyl-, 1-hexahydroazepino- or a 1-piperazinyl-ring of formula (c) 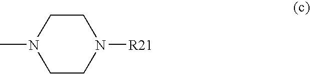
wherein
R21 is 1-4C-alkyl, pyrid-4-yl, pyrid-4-ylmethyl, dimethylamino-1-4C-alkyl, dimethylaminocarbonylmethyl, N-methyl-piperidin-4-yl, 4-morpholino-ethyl or tetrahydrofuran-2-ylmethyl,

R18 is halogen, nitro, cyano, carboxyl, 1-4C-alkyl, trifluoromethyl, 1-4C-alkoxy, 1-4C-alkoxycarbonyl, amino, mono-or di-1-4C-alkylamino, aminocarbonyl, 1-4C-alkylcarbonylamino or mono-or di-1-4C-alkylaminocarbonyl,

R19 is halogen, amino, nitro, 1-4C-alkyl or 1-4C-alkoxy,

R20 is halogen,

Aryl1 is pyrimidin-2-yl, thieno-[2,3-d]pyrimidin-4-yl, 1-methyl-1H-pyrazolo-[3,4-d]pyrimidin-4-yl, thiazolyl, imidazolyl, furanyl, pyridyl, phenyl or phenyl substituted by R18 and/or R19,

Aryl2 is pyridyl, phenyl, phenyl substituted by R18 and/or R19, 2-oxo-2H-chromen-7-yl or 4-(1,2,3-thiadiazol-4-yl)phenyl,

n is an integer from 1 to 4,

m is an integer from 1 to 4,

and the salts of these compounds.

Compounds of formula 1 of embodiment C to be emphasized are those in which

R1 is hydrogen,

R2 is hydrogen or 1-4C-alkyl,

R3 represents a phenyl derivative of formulae (a) or (b) 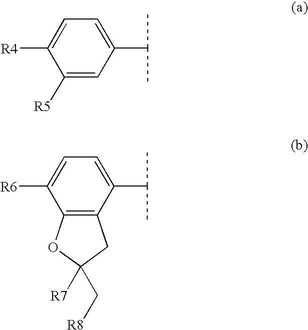
wherein
R4 is 1-2C-alkoxy or 1-2C-alkoxy which is completely or predominantly substituted by fluorine,
R5 is 1-4C-alkoxy,
R6 is 1-2C-alkoxy or 1-2C-alkoxy which is completely or predominantly substituted by fluorine,
R7 is methyl and
R8 is hydrogen,
or wherein
R7 and R8 together and with inclusion of the two carbon atoms, to which they are bonded, form a spiro-linked cyclopentane, cyclohexane, tetrahydrofurane or tetrahydropyran ring,

R9 is hydrogen, 1-4C-alkyl, —S(O)₂—R10, —C(O)R13, —(CH₂)ₘ—C(O)—R15, Aryl1 or (Aryl2)-1-4C-alkyl,

R10 is 1-4C-alkyl, 5-dimethylaminonaphthalin-1-yl or —N(R16)R17,

R13 is 1-4C-alkyl, phenyl, 4-ethyl-piperazin-2,3-dion-1-yl or —N(R16)R17,

R15 is —N(R16)R17, phenyl or phenyl substituted by R18 and/or R19 and/or R20,

R16 and R17 are independent from each other hydrogen, 1-7C-alkyl, 3-7C-cycloalkyl, 3-7C-cycloalkyl-methyl, phenyl or phenyl substituted by R18 and/or R19 and/or R20, or R16 and R17 together and with inclusion of the nitrogen atom to which they are bonded, form a 4-morpholinyl-, 1-pyrrolidinyl-, 1-piperidinyl-, 1-hexahydroazepino- or a 4-methyl-piperazin-1-yl-ring,

R18 is halogen, nitro, cyano, 1-4C-alkyl, trifluoromethyl, 1-4C-alkoxy or 1-4C-alkoxycarbonyl,

R19 is halogen, amino, nitro, 1-4C-alkyl or 1-4C-alkoxy,

R20 is halogen,

Aryl1 is pyrimidin-2-yl, thieno-[2,3-d]pyrimidin-4-yl, 1-methyl-1H-pyrazolo-[3,4-d]pyrimidin-4-yl, pyridyl, phenyl or phenyl substituted by R18 and/or R19,

Aryl2 is pyridyl, phenyl, phenyl substituted by R18 and/or R19, 2-oxo-2H-chromen-7-yl or 4-(1,2,3-thiadiazol-4-yl)phenyl,

m is an integer from 1 to 2,

and the salts of these compounds.

Compounds of formula 1 of embodiment C particularly to be emphasized are those in which

R1 is hydrogen,

R2 is hydrogen or methyl,

R3 represents a phenyl derivative of formulae (a) or (b) 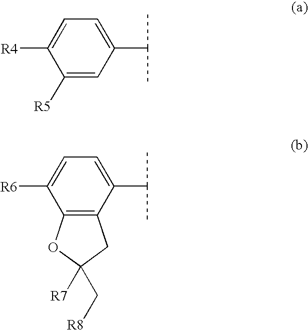
whereih
R4 is 1-2C-alkoxy,
R5 is 1-4C-alkoxy,
R6 is 1-2C-alkoxy,
R7 is methyl and
R8 is hydrogen,

R9 is hydrogen, —S(O)₂—R10, —C(O)R13, —(CH₂)ₘ—C(O)—R15 or (Aryl2)-1-2C-alkyl,

R10 is 1-4C-alkyl, 5-dimethylaminonaphthalin-1-yl or —N(R16)R17,

R13 is 1-4C-alkyl, 4-ethyl-piperazin-2,3-dion-1-yl or —N(R16)R17,

R15 is —N(R16)R17,

R16 and R17 are independent from each other hydrogen or 1-4C-alkyl, or R16 and R17 together and with inclusion of the nitrogen atom to which they are bonded, form a 4-morpholinyl ring, a 1-piperidinyl ring or a 4-methyl-piperazin-1-yl ring,

Aryl2 is pyridyl or phenyl,

m is 1,

and the salts of these compounds.

Preferred compounds of formula 1 of embodiment C are those in which

R1 is hydrogen,

R2 is methyl,

R3 represents a phenyl derivative of formula (a) 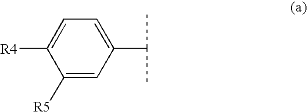
wherein
R4 is methoxy,
R5 is methoxy and

R9 is acetyl, morpholin-4-ylcarbonyl, pyridin-3-ylmethyl, 4-ethyl-piperazin-2,3-dion-1-yl, 4-methylpiperazin-1-yl, 5-dimethylamino-naphythalene-1-sulfonyl or morpholin4-yl-2-oxo-ethyl,

and the salts of these compounds.

A special embodiment of the compounds of the present invention include those compounds of formula 1 in which R3 represents a phenyl derivative of formula (a).

Another special embodiment of the compounds of the present invention include those compounds of formula 1 in which R3 represents a phenyl derivative of formula (a) and R4 and R5 have the meaning methoxy.

A further special embodiment of the compounds of the present invention include those compounds of formula 1 in which R1 is hydrogen, R2 is hydrogen or methyl, R3 represents a phenyl derivative of formula (a) and R4 and R5 have the meaning methoxy.

The compounds of formula 1 are can be chiral compounds. Chiral centers exist in the compounds of formula 1 in positions 4 and 5 of the pyridazinone ring, if R1 and/or R2 have a meaning other than hydrogen. In case R3 represents a phenyl derivative of formula (b) there is one further chiral center in the dihydrofuran-ring, if the substituents —R7 and —CH₂R8 are not identical. However, preferred are in this connection those compounds, in which the substituents —R7 and —CH₂R8 are identical or together and with inclusion of the two carbon atoms to which they are bonded form a spiro-connected 5-, 6- or 7-membered hydrocarbon ring.

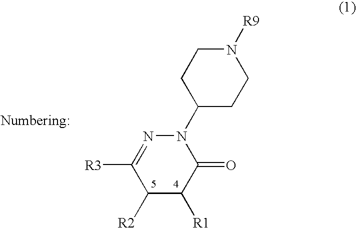

The invention includes all conceivable pure diastereomers and pure enantiomers of the compounds of formula 1, as well as all mixtures thereof independent from the ratio, including the racemates.

The compounds according to the invention can be prepared, for example, as described in Reaction scheme 1.

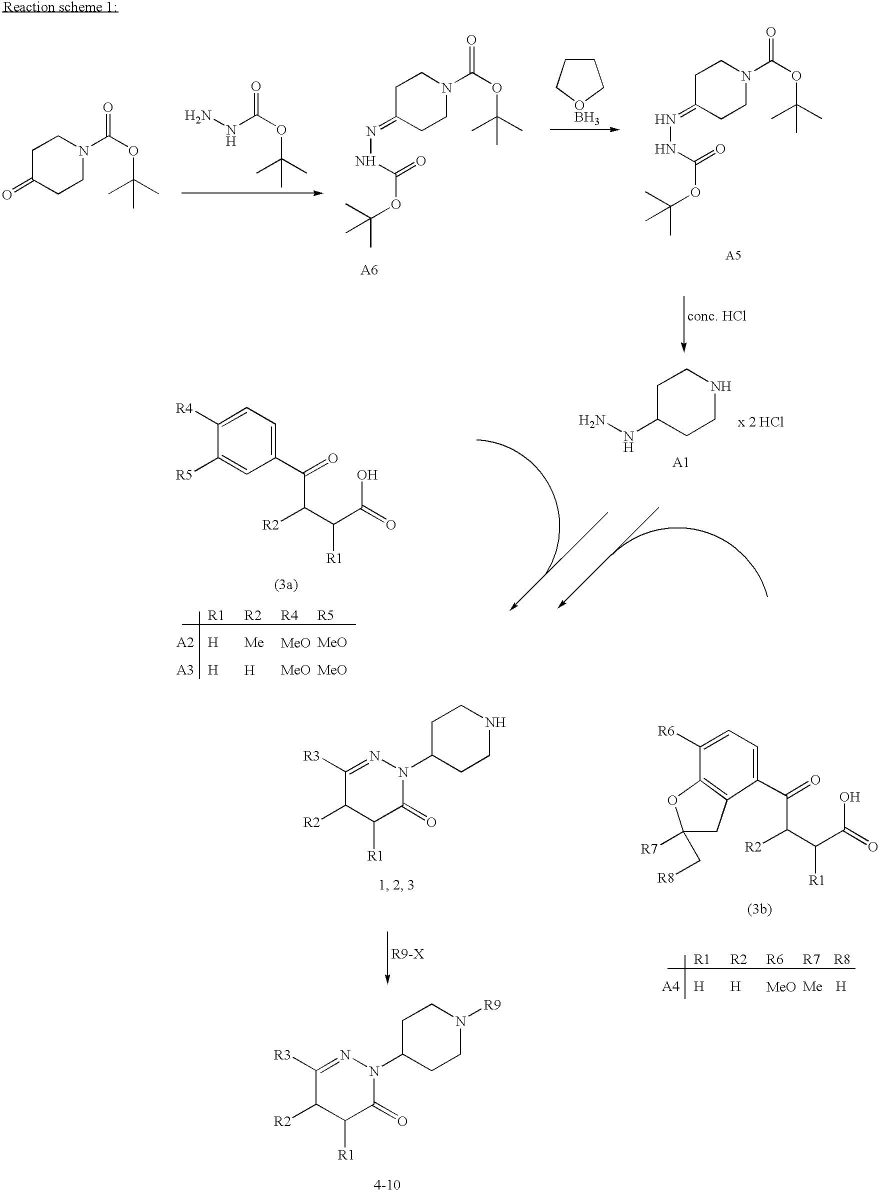

Reaction scheme 1 shows that the compounds of formula 1 can be, for example, prepared starting from 4-oxo-piperidine-1-carboxylic acid tert-butyl ester which is reacted in a first reaction step with tertbutylcarbazate to give 4-(tert-Butoxycarbonyl-hydrazono)-piperidine-1-carboxylic acid tert-butyl ester (starting compound A6). Compound A6 is reduced with, for example, the boran tetrahydrofurane complex to give 4-(N′-tert-Butoxycarbonyl-hydrazino)-piperidine-1-carboxylic acid tert-butyl ester (starting compound A5). Treatment of compound A5 with concentrated hydrochloric acid results in the formation of piperidin-4-yl-hydrazine dihydrochloride (starting compound A1).

The reaction of piperidin-4-yl-hydrazine dihydrochloride with phenyl-4-oxo-butyric acids of formulae 3a or 3b leads to the piperidino derivatives of formula 2.

These are reacted in the final reaction step with compounds of formula R9-X, wherein X represents a suitable leaving group, preferably a chlorine atom, to give the compounds of formula 1.

Suitably, the conversions are carried out analogous to methods which are familiar per se to the person skilled in the art, for example, in the manner which is described in the following examples.

The preparation of the phenyl-4-oxo-butyric acids of formulae 3a or 3b is known to the person skilled in the art (see for example Starting compounds and Intermediates).

The preparation of compounds of formula R9-X is also known to the person skilled in the art.

The substances according to the invention are isolated and purified in a manner known per se, e.g. by distilling off the solvent in vacuo and recrystallising the residue obtained from a suitable solvent or subjecting it to one of the customary purification methods, such as column chromatography on a suitable support material.

Salts are obtained by dissolving the free compound in a suitable solvent (for example a ketone like acetone, methylethylketone, or methylisobutylketone, an ether, like diethyl ether, tetrahydrofuran or dioxane, a chlorinated hydrocarbon, such as methylene chloride or chloroform, or a low molecular weight aliphatic alcohol, such as ethanol, isopropanol) which contains the desired acid, or to which the desired acid is then added. The salts are obtained by filtering, reprecipitating, precipitating with a non-solvent for the addition salt or by evaporating the solvent. Salts obtained can be converted by basification into the free compounds which, in turn, can be converted into salts. In this manner, pharmacologically non-tolerable salts can be converted into pharmacologically tolerable salts.

The following examples illustrate the invention in greater detail, without restricting it. As well, further compounds of formula 1, of which the preparation is explicitly not described, can be prepared in an analogous way or in a way which is known by a person skilled in the art using customary preparation methods.

The compounds, which are mentioned in the examples as well as their salts are preferred compounds of the invention. In the examples, RT stands for room temperature, h for hour(s), min for minute(s) and M. p. for melting point.

### EXAMPLES

Final Products

### 1. 6-(3,4-Dimethoxy-phenyl)-5-methyl-2-piperidin-4-yl-4,5-dihydro-2H-pyridazin-3-one hydrochloride

A mixture of 50 mmol of starting compound A1, 50 mmol of starting compound A2 and 100 mmol of triethylamine in 100 ml of 1-propanol is refluxed for 18 h and subsequently evaporated. The residue is partitioned between dichloromethane and aqueous sodium carbonate. The dichloromethane solution is dried over magnesium sulfate. Addition of a saturated solution of hydrochloric acid in diethyl ether causes precipitation of the title compound. M. p. 91-95° C.

### 2. 6-(3,4-Dimethoxy-phenyl)-2-piperidin-4-yl-4,6-dihydro-2H-pyridazin-3-one hydrochloride

Prepared as described for compound 1 from starting compounds A1 and A3. M. p. 227-229° C.

### 3. 6-(7-Methoxy-2,2-dimethyl-2,3-dihydro-benzofuran-4-yl)-2-piperidin-4-yl-4,5-dihydro-pyridazin-3-one hydrochloride

Prepared as described for compound 1 from starting compounds A1 and A4. M. p. 280° C. (with decomposition)

### 4. 2-(1-Acetyl-piperidin-4-yl)-6-(3,4-dimethoxy-phenyl)-5-methyl-4,5-dihydro-2H-pyridazin-3-one

To a solution of 5 mmol of compound 1 and 20 mmol of triethylamine in 50 ml of dichloromethane, 10 mmol of acetic anhydride is added and the resulting mixture is stirred at RT. After 60 min the solution is washed subsequently with diluted hydrochloric acid and aqueous sodium carbonate. The solution is dried over magnesium sulfate and evaporated. The residue is crystallized from diethyl ether. M. p. 149-152° C.

### 5,6-(3,4-Dimethoxy-phenyl)-5-methyl-2-[1-(1-morpholin-4-yl-methanoyl)-piperidin-4-yl-4,5-dihydro-2H-pyridazin-3-one

Prepared from compound 1 and morpholine4-carbonyl chloride as described for compound 4. M. p. 137-138° C.

### 6. 6-(3,4-Dimethoxy-phenyl)-5-methyl-2-(1-pyridin-3-ylmethyl-piperidin-4-yl)-4,5-dihydro-2H-pyridazin-3-one dihydrochloride

A mixture of 5 mmol of compound 1, 7 mmol of 3-chloromethyl-pyridine hydrochloride and 20 mmol of potassium carbonate in 20 ml of dimethylformamide is stirred at RT for 18 h. After addition of 150 ml of water, the mixture is extracted with diethyl ether. The ether solution is dried over magnesium sulfate and evaporated. The residue is purified by chromatography (elution with a mixture of ethyl acetate and methanol, 3:1). Addition of a saturated solution of hydrochloric acid in ether to the purified fractions causes precipitation of the title compound. M. p. 241-244° C.

### 7. 1-(1-4-[3-(3,4-Dimethoxy-phenyl)-6-oxo-5,6-dihydro-4H-pyridazin-1-yl]-piperidin-1-yl}-methanoyl)-4-ethyl-piperazine-2,3-dione

Prepared from compound 2 and 4-ethyl-2,3-dioxo-piperazine-1-carbonyl chloride as described for compound 4. M. p. 201-203° C.

### 8. 6-(3,4-Dimethoxy-phenyl)-2-{1-[1-(4-methyl-piperazin-1-yl)-methanoyl]-piperidin-4-yl}-4,5-dihydro-2H-pyridazin-3-one hydrochloride

Prepared from compound 2 and 4-methyl-piperazine-1-carbonyl chloride as described for compound 4. After evaporating the dichloromethane solution, the residue is dissolved in ethyl acetate and addition of a saturated solution of hydrochloric acid in ether causes precipitation of the title compound. Recrystallisation is performed from a mixture of methanol and ethyl acetate. M. p. 151-154° C.

### 9. 6-(3,4-Dimethoxy-phenyl)-2-[1-(5-dimethylamino-naphthalene-1-sulfonyl)-piperidin-4-yl]-4,5-dihydro-2-pyridazin-3-one

Prepared from compound 2 and 5-dimethylamino-naphthalene-1-sulfonyl chloride as described for compound 4. M. p. 191-193° C.

### 10. 6-(3,4-Dimethoxy-phenyl)-2-[1-(2-morpholin4-yl-2-oxo-ethyl)-piperidin-4-yl]-4,5-dihydro-2-pyridazin-3-one hydrochloride

Prepared from compound 2 and 2-chloro-1-morpholin-4-yl-ethanone as described for compound 6. M. p. 145-148° C.

### 11. 6-(3,4-Dimethoxy-phenyl)-2-[1-(toluene-4-sulfonyl)-piperidin-4-yl]-4,5-dihydro-2H-pyridazin-3-one

Prepared from 4-toluenesulfonyl chloride and compound 1 as described for compound 4. M. p. 179-184° C.

### 12. 6-(3,4-Dimethoxy-phenyl)-2-(1-methanesulfonyl-piperidin-4-yl)-4,5-dihydro-2H-pyridazin-3-one

Prepared from methylsulfonyl chloride and compound 1 as described for compound 4. M. p. 164-166° C.

### 13. 2-[1-(4-Chloro-benzenesulfonyl)-piperidin-4-yl]-6-(3,4-dimethoxy-phenyl)-4,5-dihydro-2H-pyridazin-3-one

Prepared from 4-chloro-benzenesulfonyl chloride and compound 1 as described for compound 4. M. p. 185-186° C.

### 14. 6-(3,4-Dimethoxy-phenyl)-2-(1-phenylmethanesulfonyl-piperidin-4-yl)-4,5-dihydro-2H-pyridazin-3-one

Prepared from phenylmethanesulfonyl chloride and compound 1 as described for compound 4. M. p. 114° C.

### 15. 6-(3,4-Dimethoxy-phenyl)-2-[1-(4-methoxy-benzenesulfonyl)-piperidin-4-yl]-4,5-dihydro-2H-pyridazin-3-one

Prepared from 4-methoxy-benzenesulfonyl chloride and compound 1 as described for compound 4. M. p. 197-198° C.

### 16. 2-(1-Benzenesulfonyl-piperidin-4-yl)-6-(3,4-dimethoxy-phenyl-4,5-dihydro-2H-pyridazin-3-one

Prepared from benzenesulfonyl chloride and compound 1 as described for compound 4. M. p. 188-190° C.

### 17. 2-[1-(2,5-Dimethoxy-benzenesulfonyl)-piperidin-4-yl-6-(3,4-dimethoxy-phenyl)-4,5-dihydro-2H-pyridazin-3-one

Prepared from 2,5-Dimethoxy-benzenesulfonyl chloride and compound 1 as described for compound 4. M. p. 184-185° C.

### 18. 2-{4-[3-(3,4-Dimethoxy-phenyl)-6-oxo-5,6-dihydro-4H-pyridazin-1-yl]-piperidine-1-sulfonyl}-benzonitrile

Prepared from 2-cyano-benzenesulfonyl chloride and compound 1 as described for compound 4. M. p. 158-160° C.

### 19. 6-(3,4-Dimethoxy-phenyl)-2-[1-(thiophene-2-sulfonyl)-piperidin-4-yl]-45-dihydro-2H-pyridazin-3-one

Prepared from 2-thiophene sulfonyl chloride and compound 1 as described for compound 4. M. p. 178-179° C.

### 20. 6-(3,4-Dimethoxy-phenyl)-2-[1-(2-fluoro-benzenesulfonyl)-Piperidin-4-yl]-4,5-dihydro-2H-pyridazin-3-one

Prepared from 2-fluoro-benzenesulfonyl chloride and compound 1 as described for compound 4. M. p. 198-199° C.

### 21. 6-(3,4-Dimethoxy-phenyl)-2-[1-(2-trifluoromethoxy-benzenesulfonyl)-piperidin-4-yl]-4,5-dihydro-2H-pyridazin-3-one

Prepared from 2-trifluoromethoxy-benzenesulfonyl chloride and compound 1 as described for compound 4. M. p. 118-119° C.

### 22. 4-[3-(3,4-Dimethoxy-phenyl)-6-oxo-5,6-dihydro-4H-pyridazin-1-yl]-piperidine-1-sulfonic acid dimethylamide

Prepared from dimethylsulfamoyl chloride and compound 1 as described for compound 4. M. p. 103-106° C.

### 23. 6-(3,4-Dimethoxy-phenyl)-2-[1-(1-phenyl-methanoyl)-piperidin-4-yl]-4,5-dihydro-2H-pyridazin-3-one

Prepared from benzoyl chloride and compound 1 as described for compound 4. M. p. 152-154° C.

### 24. 6-(3,4-Dimethoxy-phenyl)-2-[1-(1-pyridin-3-yl-methanoyl)-piperidin-4-yl]-4,5-dihydro-2H-pyridazin-3-one

Prepared from nicotinoyl chloride and compound 1 as described for compound 4. M. p. 162-164° C.

### 25. 6-(3,4-Dimethoxy-phenyl)-2-{1-[1-(2,4,6-trichloro-phenyl)-methanoyl]-piperidin-4-yl}-4,5-dihydro-2H-pyridazin-3-one

Prepared from 2,4,6-trichlorobenzoyl chloride and compound 1 as described for compound 4. M. p. 156-159° C.

### 26. 4-[3-(3,4-Dimethoxy-phenyl)-6-oxo-5,6-dihydro-4H-pyridazin-1-yl]-piperidine-1-carboxylic acid tert-butylamide

Prepared from tert-butyl isocyanate and compound 1 as described for compound 4. M. p. 76-78° C.

### 27. 2-{4-[3-(3,4-Dimethoxy-phenyl)-6-oxo-5,6-dihydro-4H-pyridazin-1-yl]-piperidin-1-yl}-N,N-dimethylacetamide hydrochloride

Prepared from 2-chloro-N,N-dimethylacetamide and compound 1 as described for compound 6. M. p. 121-122° C.

### 28. 6-(3,4-Dimethoxy-phenyl)-2-{1-[2-(4-methyl-piperazin-1-yl)-2-oxo-ethyl]-piperidin-4-yl}-4,5-dihydro-2H-pyridazin-3-one dihydrochloride

Prepared from 2-chloro-1-(4-methyl-piperazin-1-yl)-ethanone hydrochloride and compound 1 as described for compound 6. M. p. 194-199° C.

### 29. 6-(3,4-Dimethoxy-phenyl)-2-{1-[2-(4-pyridin-4-yl-piperazin-1-yl)-ethanoyl]-piperidin-4-yl}-4,5-dihydro-2H-pyridazin-3-one dihydrochloride

Prepared from 1-Pyridin-4-yl-piperazine and starting compound A7 as described for compound 6. M. p. 98-99° C.

### 30. 6-(3,4-Dimethoxy-phenyl)-2-(1-{2-[4-(2-methoxy-phenyl)-piperazin-1-yl]-ethanoyl}-piperidin-4-yl)-4,5-dihydro-2H-pyridazin-3-one dihydrochloride

Prepared from 1-(2-methoxy-phenyl)-piperazine and compound A7 as described for compound 6. M. p. 109-110° C.

### 31. 6-(3,4-Dimethoxy-phenyl)-2-(1-{2-[4-(1,1-diphenyl-methyl)-piperazin-1-yl]-ethanoyl}-piperidin-4-yl)-4,5-dihydro-2H-pyridazin-3-one dihydrochloride

Prepared from 1-(1,1-diphenyl-methyl)-piperazine and compound A7 as described for compound 6. M. p. 126-127° C.

Starting Compounds and Intermediates

A1. Piperidin-4-yl-hydrazine dihydrochloride

A mixture of 0.1 mole of 4-(N′-tert-Butoxycarbonyl-hydrazino)-piperidine-1-carboxylic acid tert-butyl ester (starting compound A5) and 150 ml of concentrated hydrochloric acid is heated at 90° C. for 60 min after which the clear solution is evaporated. The residue is washed with tetrahydrofurane, filtered off and dried under vacuum. M. p. 256-259° C.

### A2. 4-(3,4-Dimethoxy-thenyl)-3-methyl-4-oxo-butyric acid

Prepared according to Haworth and Woodcock; J. Chem. Soc. 1938, 809-811

### A3. 4-(3,4-Dimethoxy-phenyl)-4-oxo-butyric acid

Prepared according to M. S. Y. Khan and Anees A. Siddiqui; Indian J. Chem. Section B, 2000, 39, 614-619

### A4. 4-(7-Methoxy-2,2-dimethyl-2,3-dihydro-benzofuran-4-yl)-4-oxo-butyric acid

Prepared analogously to (cis)-2-(2,3-Dihydro-7-methoxybenzofuran-2-spiro-1′-cyclopentane-4-carbon-yl)-1,2,3,6-tetrahydrobenzoic acid as described in WO99/31090 starting from 4-bromo-7-methoxy-2,2-dimethyl-2,3-dihydro-benzofuran and succinic anhydride. M. p. 125-126° C.

### A5. 4-(N′-tert-Butoxycarbonyl-hydrazino)-piperidine-1-carboxylic acid tert-butyl ester

150 ml of a solution of borohydride in tertahydrofurane (1.0 mol/l) is slowly added to a solution of 0.12 mole of 4-(tert-Butoxycarbonyl-hydrazono)-piperidine-1-carboxylic acid tert-butyl ester (starting compound A6) in 100 ml of dry tetrahydrofurane. After complete addition, the mixture is stirred for another 30 min after which a 100 ml of water is added to destroy the excess of borohydride. Subsequently the tetrahydrofurane is evaporated and the resulting aqeous solution is extracted with diethyl ether. After drying the solvent over magnesium sulfate, the ether is evaporated. M. p.112-115° C.

### A6. 4-(tert-Butoxycarbonyl-hydrazono)-piperidine-1-carboxylic acid tert-butyl ester

A mixture of 0.15 mole of 4-oxo-piperidine-1-carboxylic acid tert-butyl ester and 0.15 mole of tertbutylcarbazate in 250 ml of hexane is stirred for 18 h at RT. The precipitate is filtered off and dried under vacuum. M. p. 172-174° C.

### A7. 2-[1-(2-Chloro-ethanoyl)-Piperidin-4-yl]-6-(3,4-dimethoxy-phenyl)-4,5-dihydro-2H-pyridazin-3-one

A solution of 20 mmol of chloroacetyl chloride in 50 ml of dichloromethane is added slowly to a solution of 15 mmol of compound 1 and 40 mmol of triethylamine in 150 ml of dichloromethane at 0° C. After complete addition, the mixture is washed successively with diluted hydrochloric acid and with aqueous sodium carbonate, dried over magnesium sulfate and evaporated. The compound is crystallized from ethyl acetate. M. p. 1 16-117° C.

### Commercial Utility

The compounds according to the invention have valuable pharmacological properties which make them commercially utilizable. As selective inhibitors of type 4 or type 3 and 4 of cyclic nucleotide phosphodiesterase (PDE4, PDE3/4), they are suitable on the one hand as bronchial therapeutics (for the treatment of airway obstructions on account of their dilating action and cilia-stimulating action but also on account of their respiratory rate- and respiratory drive-increasing action), but on the other hand especially for the treatment of disorders of inflammatory nature, e.g. of the airways (asthma prophylaxis), of the skin, of the intestine, of the eyes and of the joints, which are mediated by mediators such as interferons, members of the tumour necrosis factor family, interleukins, chemokines, colony-stimulating factors, growth factors, lipid mediators (e.g., inter alia, PAF, platelet-activating factor), bacterial factors (e.g. LPS), immunoglobulins, oxygen free radicals and related free radicals (e.g. nitrogen monoxide NO), biogenic amines (e.g. histamine, serotonin), kinins (e.g. bradykinin), neurogenic mediators (such as substance P, neurokinin), proteins such as, for example, granular contents of leukocytes (inter alia cationic proteins of eosinophils) and adherence proteins (e.g. integrins). The compounds according to the invention have smooth muscle-relaxant action, e.g. in the region of the bronchial system, of the blood circulation, and of the efferent urinary passages. Furthermore, they have cilia frequency-increasing action, for example in the bronchial system.

On account of their PDE-inhibiting properties, the compounds according to the invention can be employed as therapeutics in human and veterinary medicine, where they can be used, for example, for the treatment and prophylaxis of the following diseases: acute and chronic (in particular inflammatory and allergen-induced) respiratory disorders of various origins (bronchitis, allergic bronchitis, bronchial asthma, emphysema, COPD); disorders associated with impaired cilia function or increased demands on ciliar clearance (bronchitis, mucoviscidosis), dermatoses (especially of proliferative, inflammatory and allergic type) such as, for example, psoriasis (vulgaris), toxic and allergic contact eczema, atopic eczema, seborrheic eczema, lichen simplex, sunburn, pruritus in the anogenital area, alopecia areata, hypertrophic scars, discoid lupus erythematosus, follicular and widespread pyodermias, endogenous and exogenous acne, acne rosacea and other proliferative, inflammatory and allergic skin disorders; disorders which are based on excessive release of TNF and leukotrienes, i.e., for example, disorders of the arthritis type (rheumatoid arthritis, rheumatoid spondylitis, osteoarthritis and other arthritic conditions), systemic lupus erythematosus, disorders of the immune system (AIDS), including AIDS-related encephalopathies, autoimmune disorders such as diabetes mellitus (type 1, autoimmune diabetes), multiple sclerosis and of the type virus-, bacteria- or parasite-induced demyelinization diseases, cerebral malaria or Lyme's disease, shock symptoms [septic shock, endotoxin shock, Gram-negative sepsis, toxic shock syndrome and ARDS (adult respiratory distress syndrome)] and also generalized inflammations in the gastrointestinal region (Crohn's disease and ulcerative colitis); disorders which are based on allergic and/or chronic, faulty immunological reactions in the region of the upper airways (pharynx, nose) and of the adjacent regions (paranasal sinuses, eyes), such as, for example, allergic rhinitis/sinusitis, chronic rhinitis/sinusitis, allergic conjunctivitis and also nasal polyps; and also disorders of the central nervous system such as memory disorders and Alzheimer's disease, candidiasis, leishmaniases and leprosy.

On account of their vasorelaxant activity, the compounds according to the invention can also be used for the treatment of high blood pressure disorders of various origins such as, for example, pulmonary high blood pressure and the concomitant symptoms associated therewith, for the treatment of erectile dysfunction or colics of the kidneys and the ureters in connection with kidney stones.

On account of their cAMP-increasing action, however, they can also be used for disorders of the heart which can be treated by PDE inhibitors, such as, for example, cardiac insufficiency, and also as anti-thrombotic, platelet aggregation-inhibiting substances.

The invention further relates to a method for the treatment of mammals including humans who are suffering from one of the abovementioned diseases. The method comprises administering a therapeutically effective and pharmacologically acceptable amount of one or more of the compounds according to the invention to the sick mammal.

The invention further relates to the compounds according to the invention for use in the treatment and/or prophylaxis of diseases, in particular the diseases mentioned.

The invention also relates to the use of the compounds according to the invention for the production of pharmaceutical compositions which are employed for the treatment and/or prophylaxis of the diseases mentioned.

The invention furthermore relates to pharmaceutical compositions for the treatment and/or prophylaxis of the diseases mentioned and which contain one or more of the compounds according to the invention.

A further subject of the invention is a commercial product, consisting of a customary secondary pack, a primary pack containing the pharmaceutical composition (for example an ampoule or a blister pack) and, if desired, an information leaflet, the pharmaceutical composition exhibiting antagonistic action against cyclic nucleotide phosphodiesterases of type 4 or types 3 and 4 and leading to the attenuation of the symptoms of illnesses which are connected with cyclic nucleotide phosphodiesterases of type 4 or types 3 and 4, and the suitability of the pharmaceutical composition for the prophylaxis or treatment of illnesses which are connected with cyclic nucleotide phosphodiesterases of type 4 or types 3 and 4 being indicated on the secondary pack and/or on the information leaflet of the commercial product, and the pharmaceutical composition containing one or more compounds of formula 1 according to the invention. The secondary pack, the primary pack containing the pharmaceutical composition and the information leaflet otherwise comply with what would be regarded as standard to the person skilled in the art for pharmaceutical compositions of this type.

Advantageously, the substances according to the invention are also suitable for combination with other substances which bring about stimulation of cAMP, such as prostaglandins (PGE2, PGI2 and prostacyclin) and their derivatives, direct adenylate cyclase stimulators such as forskolin and related substances, or substances indirectly stimulating adenylate cyclase, such as catecholamines and adrenergic receptor agonists, in particular beta-mimetics. In combination, on account of their cAMP degradation-inhibiting action, they in this case display a synergistic, superadditive activity. This comes to bear, for example, in their use in combination with PGE2 for the treatment of pulmonary hypertension.

The pharmaceutical compositions are prepared by processes which are known per se and familiar to the person skilled in the art. As pharmaceutical compositions, the compounds according to the invention (=active compounds) are either employed as such, or preferably in combination with suitable pharmaceutical auxiliaries and/or excipients, e.g. in the form of tablets, coated tablets, capsules, caplets, suppositories, patches (e.g. as TTS), emulsions, suspensions, gels or solutions, the active compound content advantageously being between 0.1 and 95% and where, by the appropriate choice of the auxiliaries and/or excipients, a pharmaceutical administration form (e.g. a delayed release form or an enteric form) exactly suited to the active compound and/or to the desired onset of action can be achieved.

The person skilled in the art is familiar with auxiliaries or excipients which are suitable for the desired pharmaceutical formulations on account of his/her expert knowledge. In addition to solvents, gel formers, ointment bases and other active compound excipients, for example antioxidants, dispersants, emulsifiers, preservatives, solubilizers, colorants, complexing agents or permeation promoters, can be used.

The administration of the pharmaceutical compositions according to the invention may be performed in any of the generally accepted modes of administration available in the art. Illustrative examples of suitable modes of administration include intravenous, oral, nasal, parenteral, topical, transdermal and rectal delivery. Oral delivery is preferred.

For the treatment of disorders of the respiratory tract, the compounds according to the invention are preferably also administered by inhalation in the form of an aerosol; the aerosol particles of solid, liquid or mixed composition preferably having a diameter of 0.5 to 10 μm, advantageously of 2 to 6 μm.

Aerosol generation can be carried out, for example, by pressure-driven jet atomizers or ultrasonic atomizers, but advantageously by propellant-driven metered aerosols or propellant-free administration of micronized active compounds from inhalation capsules.

Depending on the inhaler system used, in addition to the active compounds the administration forms additionally contain the required excipients, such as, for example, propellants (e.g. Frigen in the case of metered aerosols), surface-active substances, emulsifiers, stabilizers, preservatives, flavorings, fillers (e.g. lactose in the case of powder inhalers) or, if appropriate, further active compounds.

For the purposes of inhalation, a large number of apparatuses are available with which aerosols of optimum particle size can be generated and administered, using an inhalation technique which is as right as possible for the patient. In addition to the use of adaptors (spacers, expanders) and pear-shaped containers (e.g. Nebulator®, Volumatic®)), and automatic devices emitting a puffer spray (Autohaler®), for metered aerosols, in particular in the case of powder inhalers, a number of technical solutions are available (e.g. Diskhaler®, Rotadisk®, Turbohaler® or the inhaler described in European Patent Application EP 0 505 321), using which an optimal administration of active compound can be achieved.

For the treatment of dermatoses, the compounds according to the invention are in particular administered in the form of those pharmaceutical compositions which are suitable for topical application. For the production of the pharmaceutical compositions, the compounds according to the invention (=active compounds) are preferably mixed with suitable pharmaceutical auxiliaries and further processed to give suitable pharmaceutical formulations. Suitable pharmaceutical formulations are, for example, powders, emulsions, suspensions, sprays, oils, ointments, fatty ointments, creams, pastes, gels or solutions.

The pharmaceutical compositions according to the invention are prepared by methods known per se. The dosage of the active compounds takes place in the order of magnitude customary for PDE inhibitors. Thus topical application forms (such as, for example, ointments) for the treatment of dermatoses contain the active compounds in a concentration of, for example, 0.1-99%. The dose for administration by inhalation is customarily between 0.1 and 3 mg per day. The customary dose in the case of systemic therapy (p.o. or i.v.) is between 0.01 and 10 mg per kilogram per day.

### Biological Investigations

The second messenger cyclic AMP (cAMP) is known for inhibiting inflammatory cells and cells responsible for the immunological response. The PDE4 isoenzyme is widely distributed in cells associated with the initiation and spreading of inflammatory diseases (H Tenor and C Schudt, in “Phosphodiesterase Inhibitors”, 21-40, “The Handbook of Immunopharmacology”, Academic Press 1996); its inhibition results in the increase of the intracellular cyclic AMP concentration and thus in the inhibition of cellular activation (J E Souness et al., Immunopharmacology 47: 127-162, 2000).

The anti-inflammatory potential of PDE4 inhibitors in vivo has been described in various animal models (MMTeixeira, TIPS 18: 164-170, 1997). To examine the PDE4 inhibition on a cellular level (in vitro), a large number of proinflammatory responses can be measured. Examples are the superoxide production of neutrophilic (C Schudt et al., Arch Pharmacol 344: 682-690, 1991) or eosinophilic (A Hatzelmann et al., Brit J Pharmacol 114: 821-831, 1995) granulocytes, which can be measured as luminol-enhanced chemiluminescence, or the synthesis of tumor necrosis factor alpha (TNFQ) in monocytes, macrophages or dendritic cells (Gantner et al., Brit J Pharmacol 121: 221-231, 1997 and Pulmonary Pharmacol Therap 12: 377-386, 1999). The immunomodulatory potential of the PDE4 inhibitors furthermore becomes apparent by inhibition of T-cell responses such as cytokine synthesis or proliferation (D M Essayan, Biochem Pharmacol 57: 965-973, 1999). PDE4 inhibition by the substances according to the invention is thus a central indicator of the suppression of inflammatory processes.

Some of the cells involved in inflammatory processes contain, in addition to PDE4, also the PDE3 isoenzyme which likewise contributes to the total cAMP metabolism of these cells. Examples are endothelial cells, mast cells, T-cells, macrophages and dendritic cells. In these cell types, the inhibitory action of PDE4 inhibitors can be enhanced by additional PDE3 inhibition. In the case of (respiratory) smooth muscle cells, inhibition of the PDE3 activity is furthermore important for (broncho)relaxation (A Hatzelmann et al., in “Phosphodiesterase Inhibitors”, 147-160, “The Handbook of ImmunoPharmacology”, Academic Press, 1996).

Method for Measuring Inhibition of PDE3 and PDE4 Activities

Method A:

The PDE activity was determined according to Thompson et al. (Adv Cycl Nucl Res 10: 69-92, 1979) with some modifications (Bauer and Schwabe, Naunyn-Schmiedeberg's Arch Pharmacol 311: 193-198, 1980). The test samples contained 20 mM Tris (pH 7.4), 5 mM MgCl₂, 0.5 μM cAMP or cGMP, [³H]cAMP or [³H]cGMP (about 30 000 cpm/sample), the PDE isoenzyme-specific additives described in greater detail below, the indicated concentrations of inhibitor and an aliquot of the enzyme solution in a total sample volume of 200 μl. Dilution series of the compounds according to the invention were prepared in DMSO and further diluted in the samples [1:100 (v/v)], to give the desired end concentration of the inhibitors at a DMSO concentration of 1% (v/v), which for its part has only a minute effect on PDE activity.

After preincubation at 37° C. for 5 minutes, the reaction was started by addition of the substrate (cAMP or cGMP). The samples were incubated at 37° C. for a further 15 min. The reaction was terminated by addition of 50 μl 0.2 N HCl. After cooling on ice for 10 minutes and addition of 25 μg 5′-nucleotidase (snake venom from Crotalus atrox), the mixture was again incubated at 37° C. for 10 mih and the samples were then applied to QAE Sephadex A-25 columns (sample volume 1 ml). The columns were eluted with 2 ml of 30 mM ammonium formate (pH 6.0). The radioactivity of the eluate was measured and corrected by the corresponding blank values (measured in the presence of denatured protein); the blank values were less than 5% of the total radioactivity. In no case did the proportion of hydrolyzed nucleotide exceed 30% of the original substrate concentration.

PDE3 (cGMP-inhibited) was investigated in homogenates of human platelets (see Schudt et al., Biochem Pharmacol 1991: 42, 153-162) using cAMP or cGMP as substrate.

PDE4 (cAMP-specific) was investigated in the cytosol of human polymorphonuclear leukocytes (PMNL) [isolated from leukocyte concentrates, see Schudt et al., Arch Pharmacol 1991: 344, 682-690] using cAMP as substrate. The PDE3 inhibitor motapizone (1 μM) was used to suppress the PDE3 activity emanating from contaminated platelets.

The IC₅₀ values were determined from the concentration-inhibition curves by nonlinear regression.

Method B:

The cDNA for PDE3A1 (GB no. U36798) was isolated in 2 steps using PCR. A 3′ terminal cDNA fragment of PDE3A1 was amplified from fat cells cDNA (Clontech, Palo Alto) using primers OZ 458 (5′-AAAGTCGACTCACTGGTCTGGCTTTTGG-3′) and OZ 457 (5′-GTCGACCAGGTGCCCTCGCTA-3′). The 5′ terminal cDNA fragment of PDE3A1 was amplified from Placenta cDNA (Clontech, Palo Alto) using primers OZ 455 (5′-ATGGCAGTGCCCGGCGACGCT-3′) and OZ 456 (5′-GTCGACTTTGCTTTTTAGCCT-3′). The PCR products were cloned into pCR2.1-Topo (Invitrogen, Groningen, NL) under standard conditions (the manufacturer's instructions). The 3′ fragment was cut out with HindII and cloned into the HindII site of the construct carrying the 5′ fragment. The whole ORF was subcloned into pBacPak9 (Clontech, Palo Alto) using EcoRi. Aminoacid 12 is Aspartic Acid like in sequence GB no. AJ005036, aa 69 and aa 110 are respective Serine and Glycine like in both sequences GB no. AJ005036 and GB no. M91667.

The PDE4B2 (GB no. M97515) was a gift of Prof. M. Conti (Stanford University, USA). It was amplified from the original plasmid (pCMV5) via PCR with primers Rb9 (5′-GCCAGCGTGCAAATAATGAAGG-3′) and Rb10 (5′-AGAGGGGGATTATGTATCCAC-3′) and cloned into the pCR-Bac vector (Invitrogen, Groningen, NL).

The recombinant baculovirus was prepared by means of homologous recombination in SF9 insect cells. The expression plasmids were cotransfected with Bac-N-Blue (Invitrogen, Groningen, NL) or Baculo-Gold DNA (Pharmingen, Hamburg) using a standard protocol (Pharmingen, Hamburg). Wt virus-free recombinant virus supernatants were selected using plaque assay methods. After that, high-titre virus supernatants were prepared by amplifying 3 times. PDEs were expressed in SF21 cells by infecting 2×10⁶ cells/ml with an MOI (multiplicity of infection) between 1 and 10 in serum-free SF900 medium (Life Technologies, Paisley, UK). The cells were cultured at 28° C. for 48-72 hours, after which they were pelleted for 5-10 min at 1000 g and 4° C.

The SF21 insect cells were resuspended, at a concentration of approx. 10⁷ cells/ml, in ice-cold (4° C.) homogenization buffer (20 mM Tris, pH 8.2, containing the following additions: 140 mM NaCl, 3.8 mM KCl, 1 mM EGTA, 1 mM MgCl₂, 10 mM β-mercaptoethanol, 2 mM benzamidine, 0.4 mM Pefablock, 10 μM leupeptin, 10 μM pepstatin A, 5 μM trypsin inhibitor) and disrupted by ultrasonication. The homogenate was then centrifuged for 10 min at 1000× g and the supernatant was stored at −80° C. until subsequent use (see below). The protein content was determined by the Bradford method (BioRad, Munich) using BSA as the standard.

PDE3A1 and PDE4B2 activities were inhibited by the said compounds in a modified SPA (scintillation proximity assay) test, supplied by Amersham Biosciences (see procedural instructions “phosphodiesterase [3H]cAMP SPA enzyme assay, code TRKQ 7090”), carried out in 96-well microtitre plates (MTP's). The test volume is 100 μl and contains 20 mM Tris buffer (pH 7.4), 0.1 mg of BSA (bovine serum albumin)/ml, 5 mM Mg²⁺, 0.5 μM cAMP (including about 50,000 cpm of [3H]cAMP), 1 μl of the respective substance dilution in DMSO and sufficient recombinant PDE (1000× g supernatant, see above) to ensure that 10-20% of the cAMP is converted under the said experimental conditions. The final concentration of DMSO in the assays (1% v/v) does not substantially affect the activity of the PDEs investigated. After a preincubation of 5 min at 37° C., the reaction is started by adding the substrate (cAMP) and the assays are incubated for a further 15 min; after that, they are stopped by adding SPA beads (50 μl). In accordance with the manufacturer's instructions, the SPA beads had previously been resuspended in water, but were then diluted 1:3 (v/v) in water; the diluted solution also contains 3 mM IBMX to ensure a complete PDE activity stop. After the beads have been sedimented (>30 min), the MTP's are analyzed in commercially available luminescence detection devices. The corresponding IC₅₀ values of the compounds for the inhibition of PDE activities are determined from the concentration-effect curves by means of non-linear regression.

The inhibitory values determined for the compounds according to the invention follow from the following Table 1, in which the numbers of the compounds correspond to the numbers of the examples.

The inhibitory values of the compounds 1-22 and 27 have been determined according to Method A. The inhibitory values of the compounds 23-26, 28 and 29-31 have been determined according to Method B.

TABLE 1
Inhibition of PDE4 and PDE3 acitivity
[measured as −logIC₅₀ (mol/l)]

| | PDE4 Inhibi- | PDE3 Inhibi- |
|---|---|---|
| compound | tion | tion |
| 4 | 8.00 | 5.76 |
| 5 | 7.89 | 5.75 |
| 6 | 8.39 | 5.23 |
| 7 | 8.96 | <5 |
| 8 | 7.71 | <5 |
| 9 | 7.53 | 6.61 |
| 10 | 7.17 | 5.35 |
| 11 | 8.3 | 6.3 |
| 12 | 7.8 | 6.6 |
| 14 | 8.2 | 6.6 |
| 15 | 7.8 | 6.3 |
| 17 | 7.8 | 6.8 |
| 18 | 8.0 | 7.1 |
| 19 | 8.3 | 6.9 |
| 20 | 8.4 | 6.8 |
| 21 | 8.5 | 7.0 |
| 23 | 8.1 | 6.9 |
| 25 | 8.6 | 7.8 |
| 27 | 9.2 | <5 |
| 28 | 7.7 | <5 |
| 29 | 7.8 | 5.5 |
| 31 | 7.4 | 5.4 |

1. A compound of formula 1 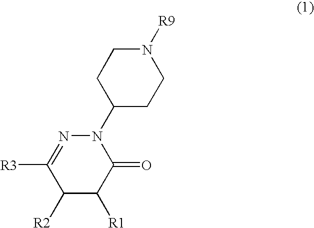
in which
R1 is hydrogen or 1-4C-alkyl,
R2 is hydrogen or 1-4C-alkyl,
R3 represents a phenyl derivative of formulae (a) or (b) 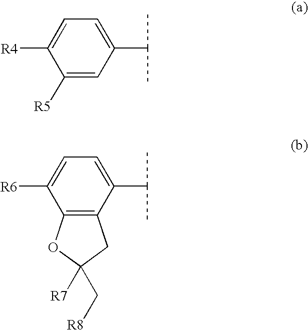
wherein
R4 is 1-4C-alkoxy or 1-4C-alkoxy which is completely or predominantly substituted by fluorine,
R5 is 1-8C-alkoxy, 3-7C-cycloalkoxy, 3-7C-cycloalkylmethoxy, or 1-4C-alkoxy which is completely or predominantly substituted by fluorine,
R6 is 1-4C-alkoxy, 3-5C-cycloalkoxy, 3-5C-cycloalkylmethoxy, or 1-4C-alkoxy which is completely or predominantly substituted by fluorine,
R7 is 1-4C-alkyl and
R8 is hydrogen or 1-4C-alkyl,
or wherein
R7 and R8 together, and with inclusion of the two carbon atoms to which they are bonded, form a spiro-linked 5-, 6- or 7-membered hydrocarbon ring, optionally interrupted by an oxygen or sulfur atom,
R9 is 1-4C-alkyl, —S(O)₂—R10, —S(O)₂—(CH₂)ₙ—R11, —(CH₂)ₘ—S(O)₂—R12, —C(O)R13, —C(O)—(CH₂)ₙ—R14, —(CH₂)ₘ—C(O)—R15, Aryl1 or (Aryl2)-1-4C-alkyl,
R10 is 1-4C-alkyl, 5-dimethylaminonaphthalin-1-yl, —N(R16)R17, thiophenyl, phenyl or phenyl substituted by R18 and/or R19,
R11 is phenyl or —N(R16)R17,
R12 is —N(R16)R17,
R13 is 1-4C-alkyl, hydroxycarbonyl-1-4C-alkyl, phenyl, 2,4,6-trichlorophenyl, pyridyl, 4-ethyl-piperazin-2,3-dion-1-yl or -N(R16)R17,
R14 is —N(R16)R17,
R15 is —N(R16)R17, phenyl or phenyl substituted by R18 and/or R19 and/or R20,
R16 is hydrogen, 1-7C-alkyl, 3-7C-cycloalkyl, 3-7C-cycloalkylmethyl, phenyl or phenyl substituted by R18 and/or R19 and/or R20,
R17 is 1-7C-alkyl, 3-7C-cycloalkyl, 3-7C-cycloalkylmethyl, phenyl or phenyl substituted by R18 and/or R19 and/or R20,
or
R16 and R17 together, and with inclusion of the nitrogen atom to which they are bonded, form a 4-morpholinyl-, 1-pyrrolidinyl-, 1-piperidinyl-, 1-hexahydroazepino- or a 1-piperazinyl-ring of formula (c) 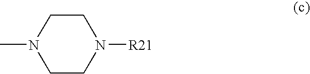
wherein
R21 is 1-4C-alkyl, pyrid-4-yl, pyrid-4-ylmethyl, 2-methoxyphenyl, 1,1-diphenylmethyl, dimethylamino-1-4C-alkyl, dimethylaminocarbonylmethyl, N-methyl-piperidin-4-yl, 4-morpholino-ethyl or tetrahydrofuran-2-ylmethyl,
R18 is halogen, nitro, cyano, carboxyl, 1-4C-alkyl, trifluoromethyl, 1-4C-alkoxy, 1-4C-alkoxy which is completely or predominantly substituted by fluorine, 1-4C-alkoxycarbonyl, amino, mono-or di-1-4C-alkylamino, aminocarbonyl, 1-4C-alkylcarbonylamino or mono-or di-1-4C-alkylaminocarbonyl,
R19 is halogen, amino, nitro, 1-4C-alkyl or 1-4C-alkoxy,
R20 is halogen,
Aryl1 is pyrimidin-2-yl, thieno-[2,3-d]pyrimidin-4-yl, 1-methyl-1H-pyrazolo-[3,4-d]pyrimidin-4-yl, thiazolyl, imidazolyl, furanyl, pyridyl, phenyl or phenyl substituted by R18 and/or R19,
Aryl2 is pyridyl, phenyl, phenyl substituted by R18 and/or R19, 2-oxo-2H-chromen-7-yl or 4-(1,2,3-thiadiazol-4-yl)phenyl,
n is an integer from 1 to 4,
m is an integer from 1 to 4,
or a hydrate, solvate, salt, hydrate of a salt or solvate of a salt thereof.

2. A compound of formula 1 according to claim 1 in which
R1 is hydrogen,
R2 is hydrogen or 1-4C-alkyl,
R3 represents a phenyl derivative of formulae (a) or (b) 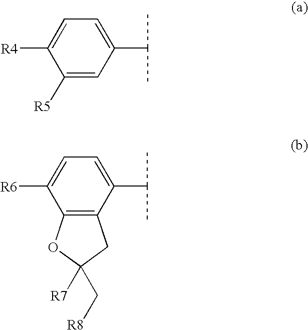
wherein
R4 is 1-2C-alkoxy or 1-2C-alkoxy which is completely or predominantly substituted by fluorine,
R5 is 1-4C-alkoxy,
R6 is 1-2C-alkoxy or 1-2C-alkoxy which is completely or predominantly substituted by fluorine,
R7 is methyl and
R8 is hydrogen,
or wherein
R7 and R8 together, and with inclusion of the two carbon atoms to which they are bonded, form a spiro-linked cyclopentane, cyclohexane, tetrahydrofurane or tetrahydropyran ring,
R9 is 1-4C-alkyl, —S(O)₂—R10, —S(O)₂—(CH₂)ₙ—R11, —C(O)R13, —C(O)—(CH₂)ₙ—R14, —(CH₂)ₘ—C(O)—R15 or (Aryl2)-1-4C-alkyl,
R10 is 1-4C-alkyl, 5-dimethylaminonaphthalin-1-yl, —N(R16)R17, thiophenyl, phenyl or phenyl substituted by R18 and/or R19,
R11 is phenyl,
R13 is 1-4C-alkyl, phenyl, pyridyl, 2,4,6-trichlorophenyl, 4-ethyl-piperazin-2,3-dion-1-yl or —N(R16)R17,
R14 is —N(R16)R17,
R15 is —N(R16)R17, phenyl or phenyl substituted by R18 and/or R19,
R16 is hydrogen, 1-7C-alkyl, 3-7C-cycloalkyl or 3-7C-cycloalkylmethyl,
R17 is 1-7C-alkyl, 3-7C-cycloalkyl or 3-7C-cycloalkylmethyl,
or
R16 and R17 together, and with inclusion of the nitrogen atom to which they are bonded, form a 4-morpholinyl-, 1-pyrrolidinyl-, 1-piperidinyl-, 1-hexahydroazepino- or a 1-piperazinyl-ring of formula (c) 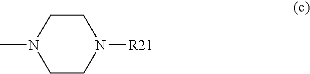
wherein
R21 is 1-4C-alkyl, pyrid-4-yl, 2-methoxyphenyl, 1,1-diphenylmethyl or N-methyl-piperidin-4-yl,
R18 is halogen, nitro, cyano, 1-4C-alkyl, trifluoromethyl, 1-4C-alkoxy, 1-4C-alkoxy which is completely or predominantly substituted by fluorine or 1-4C-alkoxycarbonyl,
R19 is halogen, amino, nitro, 1-4C-alkyl or 1-4C-alkoxy,
Aryl2 is pyridyl, phenyl or phenyl substituted by R18 and/or R19,
m is an integer from 1 to 2,
n is an integer from 1 to 2,
or a hydrate, solvate, salt, hydrate of a salt or solvate of a salt thereof.

3. A compound of formula 1 according to claim 1 in which
R1 is hydrogen,
R2 is hydrogen or methyl,
R3 represents a phenyl derivative of formula (a) 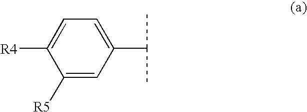
wherein
R4 is 1-2C-alkoxy,
R5 is 1-2C-alkoxy,
R9 is —S(O)₂—R10, —S(O)₂—(CH₂)ₙ—R11, —C(O)R13, —C(O)—(CH₂)ₙ—R14, —(CH₂)ₘ—C(O)—R15 or (Aryl2)-1-2C-alkyl,
R10 is 1-4C-alkyl, 5-dimethylaminonaphthalin-1-yl, —N(R16)R17, thiophenyl, phenyl or phenyl substituted by R18 and/or R19,
R11 is phenyl,
R13 is 1-4C-alkyl, phenyl, 2,4,6-trichlorophenyl, pyridyl, 4-ethyl-piperazin-2,3-dion-1-yl or —N(R16)R17,
R14 is —N(R16)R17,
R15 is —N(R16)R17,
R16 is hydrogen or 1-4C-alkyl,
R17 is 1-4C-alkyl,
or
R16 and R17 together, and with inclusion of the nitrogen atom to which they are bonded, form a 4-morpholinyl-, 1-pyrrolidinyl-, 1-piperidinyl- or a 1-piperazinyl-ring of formula (c) 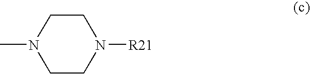
wherein
R21 is 1-4C-alkyl, pyrid-4-yl, 2-methoxyphenyl or 1,1-diphenylmethyl,
R18 is halogen, cyano, 1-4C-alkyl, trifluoromethyl, 1-4C-alkoxy or 1-4C-alkoxy which is completely or predominantly substituted by fluorine,
R19 is 1-4C-alkyl or 1-4C-alkoxy,
Aryl2 is pyridyl or phenyl,
m is 1,
n is 1,
or a hydrate, solvate, salt, hydrate of a salt or solvate of a salt thereof.

4. A compound of formula 1 according to claim 1 in which
R1 is hydrogen,
R2 is hydrogen or methyl,
R3 represents a phenyl derivative of formula (a) 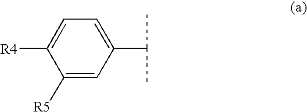
wherein
R4 is methoxy,
R5 is methoxy and
R9 is acetyl, morpholin-4-ylcarbonyl, pyridin-3-ylmethyl, 4-ethyl-piperazin-2,3-dion-1-ylcarbonyl, 4-methylpiperazin-1-ylcarbonyl, 5-dimethylamino-naphythalene-1-sulfonyl, 2-(morpholin-4-yl)-2-oxo-ethyl, 4-methylbenzenesulfonyl, methylsulfonyl, 4-chlorobenzenesulfonyl, benzylsulfonyl, 4-methoxybenzenesulfonyl, benzenesulfonyl, 2,5-dimethoxybenzenesulfonyl, 2-cyanobenzenesulfonyl, thiophen-2-ylsulfonyl, 2-fluorobenzenesulfonyl, 2-trifluoromethoxy-benzenesulfonyl, dimethylaminosulfonyl, benzoyl, pyridin-3-ylcarbonyl, 2,4,6-trichlorobenzenecarbonyl, tert-butylaminocarbonyl, dimethylaminocarbonylmethyl, 2-(4-methyl-piperazin-1-yl)-2-oxo-ethyl, 2-(4-pyridin-4-ylpiperazin-1-yl)ethanoyl, 2-[4-(2-methoxyphenyl)piperazin-1-yl]ethanoyl or 2-[4-(1,1-diphenylmethyl)piperazin-1-yl]ethanoyl,
or a hydrate, solvate, salt, hydrate of a salt or solvate of a salt thereof.

5. A compound of formula 1 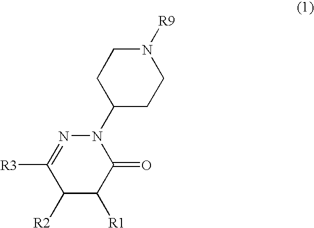
in which
R1 is hydrogen or 1-4C-alkyl,
R2 is hydrogen or 1-4C-alkyl,
R3 represents a phenyl derivative of formulae (a) or (b) 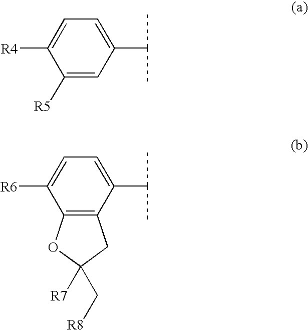
wherein
R4 is 1-4C-alkoxy or 1-4C-alkoxy which is completely or predominantly substituted by fluorine,
R5 is 1-8C-alkoxy, 3-7C-cycloalkoxy, 3-7C-cycloalkylmethoxy, or 1-4C-alkoxy which is completely or predominantly substituted by fluorine,
R6 is 1-4C-alkoxy, 3-5C-cycloalkoxy, 3-5C-cycloalkylmethoxy, or 1-4C-alkoxy which is completely or predominantly substituted by fluorine,
R7 is 1-4C-alkyl and
R8 is hydrogen or 1-4C-alkyl,
or wherein
R7 and R8 together, and with inclusion of the two carbon atoms to which they are bonded, form a spiro-linked 5-, 6- or 7-membered hydrocarbon ring, optionally interrupted by an oxygen or sulphur sulfur atom,
R9 is hydrogen, 1-4C-alkyl, —S(O)₂—R10, —S(O)₂—(CH₂)ₙ—R11, —(CH₂)ₘ—S(O)₂—R12, —C(O)R13, —C(O)—(CH₂)ₙ—R14, —(CH₂)ₘ—C(O)—R15, Aryl1 or (Aryl2)-1-4C-alkyl,
R10 is 1-4C-alkyl, 5-dimethylaminonaphthalin-1-yl, —N(R16)R17, phenyl or phenyl substituted by R18 and/or R19,
R11 is —N(R16)R17,
R12 is —N(R16)R17,
R13 is 1-4C-alkyl, hydroxycarbonyl-1-4C-alkyl, phenyl, pyridyl, 4-ethyl-piperazin-2,3-dion-1-yl or —N(R16)R17,
R14 is —N(R16)R17,
R15 is —N(R16)R17, phenyl or phenyl substituted by R18 and/or R19 and/or R20,
R16 and R17 are independent from each other hydrogen, 1-7C-alkyl, 3-7C-cycloalkyl, 3-7C-cycloalkylmethyl, phenyl or phenyl substituted by R18 and/or R19 and/or R20, or R16 and R17 together, and with inclusion of the nitrogen atom to which they are bonded, form a 4-morpholinyl-, 1-pyrrolidinyl-, 1-piperidinyl-, 1-hexahydroazepino- or a 1-piperazinyl-ring of formula (c) 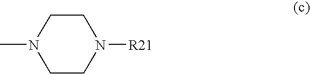
wherein
R21 is 1-4C-alkyl, pyrid-4-yl, pyrid-4-ylmethyl, dimethylamino-1-4C-alkyl, dimethylaminocarbonylmethyl, N-methyl-piperidin-4-yl, 4-morpholino-ethyl or tetrahydrofuran-2-ylmethyl,
R18 is halogen, nitro, cyano, carboxyl, 1-4C-alkyl, trifluoromethyl, 1-4C-alkoxy, 1-4C-alkoxycarbonyl, amino, mono-or di-1-4C-alkylamino, aminocarbonyl, 1-4C-alkylcarbonylamino or mono-or di-1-4C-alkylaminocarbonyl,
R19 is halogen, amino, nitro, 1-4C-alkyl or 1-4C-alkoxy,
R20 is halogen,
Aryl1 is pyrimidin-2-yl, thieno-[2,3-d]pyrimidin-4-yl, 1-methyl-1H-pyrazolo-[3,4-d]pyrimidin-4-yl, thiazolyl, imidazolyl, furanyl, pyridyl, phenyl or phenyl substituted by R18 and/or R19,
Aryl2 is pyridyl, phenyl, phenyl substituted by R18 and/or R19, 2-oxo-2H-chromen-7-yl or 4-(1,2,3-thiadiazol-4-yl)phenyl,
n is an integer from 1 to 4,
m is an integer from 1 to 4,
or a hydrate, solvate, salt, hydrate of a salt or solvate of a salt thereof.

6. A compound of formula 1 according to claim 5, in which
R1 is hydrogen,
R2 is hydrogen or 1-4C-alkyl,
R3 represents a phenyl derivative of formulae (a) or (b) 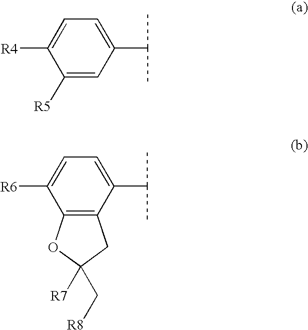
wherein
R4 is 1-2C-alkoxy or 1-2C-alkoxy which is completely or predominantly substituted by fluorine,
R5 is 1-4C-alkoxy,
R6 is 1-2C-alkoxy or 1-2C-alkoxy which is completely or predominantly substituted by fluorine,
R7 is methyl and
R8 is hydrogen,
or wherein
R7 and R8 together, and with inclusion of the two carbon atoms to which they are bonded, form a spiro-linked cyclopentane, cyclohexane, tetrahydrofurane or tetrahydropyran ring,
R9 is hydrogen, 1-4C-alkyl, —S(O)₂—R10, —C(O)R13, —(CH₂)ₘ—C(O)—R15, Aryl1 or (Aryl2)-1-4C-alkyl,
R10 is 1-4C-alkyl, 5-dimethylaminonaphthalin-1-yl or —N(R16)R17,
R13 is 1-4C-alkyl, phenyl, 4-ethyl-piperazin-2,3-dion-1-yl or —N(R16)R17,
R15 is —N(R16)R17, phenyl or phenyl substituted by R18 and/or R19 and/or R20,
R16 and R17 are independent from each other hydrogen, 1-7C-alkyl, 3-7C-cycloalkyl, 3-7C-cycloalkylmethyl, phenyl or phenyl substituted by R18 and/or R19 and/or R20, or R16 and R17 together, and with inclusion of the nitrogen atom to which they are bonded, form a 4-morpholinyl-, 1-pyrrolidinyl-, 1-piperidinyl-, 1-hexahydroazepino- or a 4-methyl-piperazin-1-yl-ring,
R18 is halogen, nitro, cyano, 1-4C-alkyl, trifluoromethyl, 1-4C-alkoxy or 1-4C-alkoxycarbonyl,
R19 is halogen, amino, nitro, 1-4C-alkyl or 1-4C-alkoxy,
R20 is halogen,
Aryl1 is pyrimidin-2-yl, thieno-[2,3-d]pyrimidin-4-yl, 1-methyl-1H-pyrazolo-[3,4-d]pyrimidin-4-yl, pyridyl, phenyl or phenyl substituted by R18 and/or R19,
Aryl2 is pyridyl, phenyl, phenyl substituted by R18 and/or R19, 2-oxo-2H-chromen-7-yl or 4-(1,2,3-thiadiazol-4-yl)phenyl,
m is an integer from 1 to 2,
or a hydrate, solvate, salt, hydrate of a salt or solvate of a salt thereof.

7. A compound of formula 1 according to claim 5, in which
R1 is hydrogen,
R2 is hydrogen or methyl,
R3 represents a phenyl derivative of formulae (a) or (b) 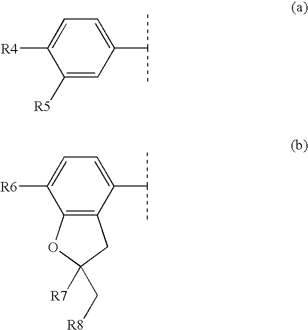
wherein
R4 is 1-2C-alkoxy,
R5 is 1-4C-alkoxy,
R6 is 1-2C-alkoxy,
R7 is methyl and
R8 is hydrogen,
R9 is hydrogen, —S(O)₂—R10, —C(O)R13, —(CH₂)ₘ—C(O)—R15 or (Aryl2)-1-2C-alkyl,
R10 is 1-4C-alkyl, 5-dimethylaminonaphthalin-1-yl or —N(R16)R17,
R13 is 1-4C-alkyl, 4-ethyl-piperazin-2,3-dion-1-yl or —N(R16)R17,
R15 is —N(R16)R17,
R16 and R17 are independent from each other hydrogen or 1-4C-alkyl, or R16 and R17 together, and with inclusion of the nitrogen atom to which they are bonded, form a 4-morpholinyl ring, a 1-piperidinyl ring or a 4-methyl-piperazin-1-yl ring,
Aryl2 is pyridyl or phenyl,
m is 1,
or a hydrate, solvate, salt, hydrate of a salt or solvate of a salt thereof.

8. A compound of formula 1 according to claim 5, in which
R1 is hydrogen,
R2 is methyl,
R3 represents a phenyl derivative of formula (a) 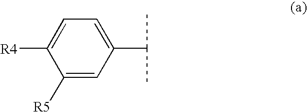
wherein
R4 is methoxy,
R5 is methoxy and
R9 is acetyl, morpholin-4-ylcarbonyl, pyridin-3-ylmethyl, 4-ethyl-piperazin-2,3-dion-1-yl, 4-methylpiperazin-1-yl, 5-dimethylamino-naphythalene-1-sulfonyl or morpholin-4-yl-2-oxo-ethyl,
or a hydrate, solvate, salt, hydrate of a salt or solvate of a salt thereof.

9. (canceled)

10. A pharmaceutical composition comprising one or more compounds of formula 1 according to claim 1, or a pharmaceutically acceptable hydrate, solvate, salt, hydrate of a salt or solvate of a salt thereof, together with a pharmaceutical auxiliary and/or excipient.

11. (canceled)

12. A method for treating an illness treatable by the administration of a PDE4 inhibitor in a patient comprising administering to said patient in need thereof a therapeutically effective amount of a compound of formula 1 as claimed in claim 1, or a pharmaceutically acceptable hydrate, solvate, salt, hydrate of a salt or solvate of a salt thereof.

13. A method for treating an airway disorder in a patient comprising administering to said patient in need thereof a therapeutically effective amount of a compound of formula 1 as claimed in claim 1, or a pharmaceutically acceptable hydrate, solvate, salt, hydrate of a salt or solvate of a salt thereof.

14. A pharmaceutical composition comprising one or more compounds of formula 1 according to claim 5, or a pharmaceutically acceptable hydrate, solvate, salt, hydrate of a salt or solvate of a salt thereof, together with a pharmaceutical auxiliary and/or excipient.

15. A method for treating an illness treatable by the administration of a PDE4 inhibitor in a patient comprising administering to said patient in need thereof a therapeutically effective amount of a compound of formula 1 as claimed in claim 5, or a pharmaceutically acceptable hydrate, solvate, salt, hydrate of a salt or solvate of a salt thereof.

16. A method for treating an airway disorder in a patient comprising administering to said patient in need thereof a therapeutically effective amount of a compound of formula 1 as claimed in claim 5, or a pharmaceutically acceptable hydrate, solvate, salt, hydrate of a salt or solvate of a salt thereof.
